(19) United States
(12) Patent Application Publication
(10) Pub. No.: US 2019/0091227 A1
(43) Pub. Date: Mar. 28, 2019

(54) METHODS FOR TREATING ER+, HER2-, HRG+ BREAST CANCER USING COMBINATION THERAPIES COMPRISING AN ANTI-ERBB3 ANTIBODY

(71) Applicant: Merrimack Pharmaceuticals, Inc., Cambridge, MA (US)

(72) Inventors: Akos CZIBERE, Medford, MA (US); Gregory J. FINN, Watertown, MA (US); Hong ZHANG, Chestnut Hill, MA (US)

(21) Appl. No.: 16/084,442

(22) PCT Filed: Mar. 15, 2017
(86) PCT No.: PCT/US2017/022517

§ 371 Date: Sep. 12, 2018

Related U.S. Application Data
(60) Provisional application No. 62/431,242, filed on Dec. 7, 2016.
(60) Provisional application No. 62/356,127, filed on Jun. 29, 2016.
(60) Provisional application No. 62/308,783, filed on Mar. 15, 2016.

(51) Int. Cl. A61K 31/519; A61K 31/4196; A61K 31/565; A61K 39/395; A61K 9/00; A61P 35/00

(52) U.S. Cl. CPC A61K 31/519; A61K 31/4196; A61K 31/565; A61K 2039/505; A61K 9/0019; A61P 35/00; A61K 39/39558

(57) ABSTRACT

Provided herein are compositions and methods for treating ER+, HER2− HRG+ breast cancer (e.g., metastatic ER+, HER2− breast cancer) in a patient by administering to the patient an anti-ErbB3 antibody (e.g., seribantumab), a CDK4/6 inhibitor (e.g., palbociclib), and an endocrine based therapy (e.g., letrozole or fulvestrant) according to a particular clinical dosage regimen (i.e., at a particular dose amount and according to a specific dosing schedule). Also provided herein are compositions and methods for treating ER+, HER2− HRG+ breast cancer (e.g., metastatic ER+, HER2− breast cancer) in a patient by administering to the patient an anti-ErbB3 antibody (e.g., seribantumab) and an endocrine based therapy (e.g., letrozole or fulvestrant) according to a particular clinical dosage regimen (i.e., at a particular dose amount and according to a specific dosing schedule).

### RELATED APPLICATIONS

This application claims priority to U.S. Provisional Application Nos. 62/308,783 filed Mar. 15, 2016, 62/356,127 filed Jun. 29, 2016 and 62/431,242, filed Dec. 7, 2016. The contents of the aforementioned applications are hereby incorporated by reference.

### BACKGROUND

Cyclin-dependent kinase 4 (CDK4) and the closely related cyclin-dependent kinase 6 (CDK6) are regulators of mammalian mitosis, acting to promote the start of DNA synthesis in preparation for cell division. Several selective inhibitors of CDKs 4 and 6 (“CDK4/6” inhibitors) are at various stages of development, with one, palbociclib, being currently approved in the United States for the treatment of hormone receptor (HR)-positive, human epidermal growth factor receptor 2 (HER2)-negative advanced or metastatic breast cancer in combination with an endocrine based therapy—either letrozole or fulvestrant. These combinations have been very effective, and are fast becoming the standard of care for such patients.

Breast cancer remains one of the most common and deadly cancers in the United States, with an expected 232,670 newly diagnosed cases and 40,000 related deaths in 2014 alone. Among these, the largest molecular diagnostic subgroup (˜70%) comprises patients with hormone receptor positive (ER+/PR+; ER+/PR−; or ER−/PR+) and HER2 negative (IHC<3+ and not FISH positive) disease. For patients with metastatic disease, the reported median survival ranges from 18-36 months. Metastatic breast cancer is not considered curable. Significant improvements in survival have been obtained, however, coincident with the introduction of improved systemic therapies.

The general therapeutic goal for all patients with ER/PR positive metastatic breast cancer is to prolong survival and improve quality of life. This is accomplished by surgical intervention, where feasible, and medication. Typically endocrine (anti-hormonal) medications are used initially, and maintained until resistance arises. These are preferred because they are effective and relatively non-toxic and their use avoids the toxicities of chemotherapy-based regimens. Current National Comprehensive Cancer Network (NCCN) treatment guidelines state: “systemic treatment of breast cancer recurrence or stage IV disease prolongs survival and enhances quality of life but is not curative. Therefore, treatments associated with minimal toxicity are preferred.”

There are several first line endocrine therapy options for ER/PR positive breast cancer patients with metastatic disease. The term “first-line” is used in this context to indicate the first line of therapy following the appearance of metastatic disease, even if patients have previously been treated in the pre-metastatic setting. In the metastatic setting, either fulvestrant or an aromatase inhibitor (AI, e.g., letrozole) is generally preferred as single agents for first-line therapy. Fulvestrant is a selective estrogen receptor down-regulator (SERD) and is indicated for the treatment of hormone receptor positive metastatic breast cancer in postmenopausal patients with disease progression following anti-estrogen therapy. Aromatase inhibitors block a key step in the synthesis of estrogen.

For postmenopausal patients with metastatic breast cancer who are endocrine therapy-naïve, have progressed >12 months after the end of adjuvant therapy, or who present with de novo metastatic breast cancer, treatment options include an AI plus palbociclib or single-agent therapy using fulvestrant or an AI.

Palbociclib (formerly PD 0332991) is an inhibitor of cyclin-dependent kinases 4 and 6 (CDK 4/6). Palbociclib plus letrozole received US Food and Drug Administration (FDA) accelerated approval as first-line therapy for the treatment of metastatic ER-positive human epidermal growth factor receptor 2 (HER2)-negative breast cancer in 2015. Treatment with letrozole plus palbociclib resulted in a statistically significant increase in progression free survival (PFS) in the combination arm. Overall survival appeared favorable of the combination arm as well, but did not reach statistical significance. There remain limited second-line and beyond endocrine therapy options in the metastatic setting with the AI, exemestane, being one of the more commonly used agents, and the SERD, fulvestrant, being another established option.

Innate and acquired resistance to endocrine therapies pose significant therapeutic challenges in this context, as only those patients who have continuous sensitivity to endocrine therapy experience long-term survival with a reasonably good quality of life. Unfortunately, many patients develop resistance to endocrine therapy, sometimes resulting in very short treatment durations, accelerating the need to initiate cytotoxic chemotherapy.

Like other kinase inhibitors used to treat cancer, the effective use of CDK4/6 inhibitors is limited by resistance—in some cases pre-existing and in most cases developing after a time on treatment. Thus a need exists for low-toxicity methods for treating patients who are resistant to CDK4/6 inhibitor treatment.

The present disclosure addresses the need for non-toxic therapies that prevent or abrogate the resistance that develops to endocrine and CDK inhibitory therapies and provides additional benefits.

### BRIEF DESCRIPTION OF THE DRAWINGS

FIG. 1. Heregulin mRNA is prevalent in human ER-positive, HER2-negative breast cancer tumors. (A) Expression of HRG mRNA extracted from the TCGA data base for ER-positive, HER2-negative breast cancer tumors. (B) Expression of HRG mRNA in 197 patient tumor samples with ER-positive, HER2-negative breast cancer, measured using HRG RNA-ISH assay where both methods found approximately 45% of samples expressed HRG mRNA.

FIG. 2. HRG promotes proliferation of ER-positive, HER2-negative breast cancer cell lines. MCF7, T47D and HCC1428 cells were stimulated with HRG for 6 days and proliferation was measured by CTG assay.

FIG. 3. HRG augments the activity of fulvestrant in ER-positive, HER2-negative breast cancer cell lines and seribantumab restores fulvestrant activity. MCF7 and T47D cells were treated with either estradiol, fulvestrant, fulvestrant and estradiol, or fulvestrant and estradiol plus seribantumab for 6 days and proliferation was measure by CTG assay.

FIG. 4. HRG inhibits the activity of CDK inhibitors in the ER-positive, HER2-negative MCF7 breast cancer cell line and seribantumab restores sensitivity. (A) MCF7 cells were treated with either palbociclib, HRG alone or in combination with seribantumab. (B) MCF7 cells were treated with either abemaciclib, HRG alone or in combination with seribantumab. (C) MCF7 cells were treated with either palbociclib, HRG alone or in combination with seribantumab. Cells were treated for 6 days and proliferation was measure by CTG assay.

FIG. 5. HRG inhibits the activity of CDK inhibitors in the ER-positive, HER2-negative ZR75-1 breast cancer cell line and seribantumab restores sensitivity. (A) ZR75-1 cells were treated with either palbociclib, HRG alone or in combination with seribantumab. (B) ZR75-1 cells were treated with either abemaciclib, HRG alone or in combination with seribantumab. (C) ZR75-1 cells were treated with either ribociclib, HRG alone or in combination with seribantumab. Cells were treated for 6 days and proliferation was measure by CTG assay.

FIG. 6. HRG inhibits the activity of CDK4/6 inhibitors in combination with fulvestrant in the ER-positive, HER2-negative MCF7 breast cancer cell line and seribantumab restores sensitivity. (A) MCF7 cells were treated with combinations of palbociclib, HRG, fulvestrant and seribantumab. (B) MCF7 cells were treated with combinations of abemaciclib, HRG, fulvestrant and seribantumab. (C) MCF7 cells were treated with combinations of ribociclib, HRG, fulvestrant and seribantumab. Cells were treated for 6 days and proliferation was measured by CTG assay.

FIG. 7. HRG inhibits the activity of CDK4/6 inhibitors in combination with tamoxifen in the ER-positive, HER2-negative MCF7 breast cancer cell line and seribantumab restores sensitivity. (A) MCF7 cells were treated with combinations of palbociclib, HRG, tamoxifen and seribantumab. (B) MCF7 cells were treated with combinations of abemaciclib, HRG, tamoxifen and seribantumab. (C) MCF7 cells were treated with combinations of ribociclib, HRG, tamoxifen and seribantumab. Cells were treated for 6 days and proliferation was measured by CTG assay.

FIG. 8. HRG activates CDK2 in MCF7 breast cancer cells and seribantumab blocks the activating effect of HRG on CDK2 activation.

FIG. 9. Fulvestrant inhibits CDK2 activation in MCF7 cells and HRG can activate CDK2 in the presence of fulvestrant. Seribantumab blocks the activating effect of HRG on CDK2 activation in the presence of fulvestrant.

FIG. 10. CDK4/6 inhibitors reduce CDK2 activation in MCF7 cells and HRG can activate CDK2 in the presence of palbociclib or abemaciclib. Seribantumab blocks the activating effect of HRG on CDK2 activation in the presence of palbociclib or abemaciclib.

FIG. 11. HRG is a highly potent ligand that inhibits the activities of fulvestrant, palbociclib and their combination in the ER-positive, HER2-negative breast cancer cells. MCF7 cells were treated with (A) fulvestrant, (B) palbociclib and (C) their combination in the presence of 1 nM of ligands for the ErbB family receptors (HRG, BTC, EGF, HB-EGF, TGF-a, AR, EPG or EPR), estrogen receptor (E2), insulin-like growth factor 1 receptor (IGF-1), c-Met (HGF), or fibroblast growth factor receptor (FGF) for 6 days and proliferation was measured by CTG assay.

FIG. 12. HRG is a highly potent ligand that inhibits the activities of fulvestrant, palbociclib and their combination in the ER-positive, HER2-negative breast cancer cells. T47D cells were treated with (A) fulvestrant, (B) palbociclib and (C) their combination in the presence of 1 nM of ligands for the ErbB family receptors (HRG, BTC, EGF, HB-EGF, TGF-a, AR, EPG or EPR), estrogen receptor (E2), insulin-like growth factor 1 receptor (IGF-1), c-Met (HGF), or fibroblast growth factor receptor (FGF) for 6 days and proliferation was measured by CTG assay.

FIG. 13. (A) HRG promotes S-phase cell cycle progression of ER+ HER2− cells and (B) HRG inhibits the activity of single agent fulvestrant and (C) single agent palbociclib or (D) the combination of palbociclib and fulvestrant on DNA synthesis and S-phase progression in ER+ positive, HER2-negative breast cancer cells. Seribantumab restores the inhibitory activity of this combination.

FIG. 14. Seribantumab addition enhances the activity of fulvestrant, palbociclib and the combination of fulvestrant and palbociclib in a human orthotopic xenograft model of ER+ HER2− breast cancer.

FIG. 15. HRG enhances the phosphorylation of retinoblastoma protein (RB) to promote cell cycle transition and inhibit the activity of fulvestrant. CDK4/6 inhibitors palbociclib or abemaciclib on RB phosphorylation and seribantumab can restore activity by blockade of HRG in a human ER+ HER2− breast cancer cells. (A) Fulvestrant inhibits RB activation of RB at Serine807/811 and HRG counteracts fulvestrant by enhancing activation of RB at Serine807/811. Seribantumab inhibits HRG to restore the activity of fulvestrant on RB activation. (B) CDK4/6 inhibitors (palbociclib and abemaciclib) decrease RB activation of RB at Serine807/811. HRG counteracts palbociclib and abemaciclib activity by enhancing activation of RB at Serine807/811. Seribantumab inhibits HRG to restore the activity of palbociclib and abemaciclib on RB activation. (C) The combination of palbociclib and fulvestrant decreases RB activation of RB at Serine807/811 and Serine 780 and HRG counteracts palbociclib and fulvestrant activity by enhancing activation of RB at Serine807/811 and Serine 780. Seribantumab inhibits HRG to restore the activity of the palbociclib-fulvestrant combination.

FIG. 16. Seribantumab and letrozole co-treatment delays the onset of resistance and restores sensitivity to letrozole in MCF-7Ca xenografts.

### SUMMARY

Provided herein are compositions and methods for treating ER+, HER2− HRG+ breast cancer (e.g., metastatic ER+, HER2− HRG+ breast cancer) in a human patient, comprising administering to the patient an anti-ErbB3 antibody (e.g., seribantumab), a CDK4/6 inhibitor (e.g., palbociclib, abemaciclib, or ribociclib), and an endocrine based therapy (e.g., letrozole or fulvestrant) according to a particular clinical dosage regimen (i.e., at a particular dose amount and according to a specific dosing schedule).

An exemplary anti-ErbB3 antibody is seribantumab (also known as “MM-121” or “Ab #6”) or antigen binding fragments and variants thereof. In one embodiment, the anti-ErbB3 antibody comprises the heavy and light chain CDRs or variable regions of seribantumab. In one embodiment, the antibody comprises the CDR1, CDR2, and CDR3 domains of the VH region of seribantumab having the sequence set forth in SEQ ID NO: 10 and the CDR1, CDR2 and CDR3 domains of the VL region of seribantumab having the sequence set forth in SEQ ID NO: 12.

In another embodiment, the antibody comprises heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. In another embodiment, the antibody comprises VH and/or VL regions having the amino acid sequences set forth in SEQ ID NO: 10 and SEQ ID NO: 12, respectively. In another embodiment, the anti-ErbB3 antibody comprises VH and/or VL regions encoded by the nucleic acid sequences set forth in SEQ ID NOs: 9 and 11, respectively. In another embodiment, the anti-ErbB3 antibody comprises heavy and/or light chains having the amino acid sequences set forth in SEQ ID NO: 7 and SEQ ID NO: 8, respectively.

In another embodiment, an antibody is used that competes for binding with and/or binds to the same epitope on human ErbB3 as the above-mentioned antibodies. In a particular embodiment, the epitope comprises residues 92-104 of human ErbB3 (SEQ ID NO: 14). In another embodiment, the epitope includes amino acid residues within positions 92-104 of human ErbB3 (SEQ ID NO: 14). In another embodiment, the antibody competes with seribantumab for binding to human ErbB3 and has at least 90% variable region amino acid sequence identity with the above-mentioned anti-ErbB3 antibodies (e.g., at least about 90%, 95% or 99% variable region identity with SEQ ID NO: 10 and SEQ ID NO: 12).

An exemplary CDK4/6 inhibitor is palbociclib. In another embodiment, the CDK4/6 inhibitor is abemaciclib. In another embodiment, the CDK4/6 inhibitor is ribociclib.

An exemplary endocrine based therapy is letrozole or fulvestrant.

Accordingly, in one aspect, methods of treating a human patient with a ER+, HER2− breast cancer are provided, the methods comprising administering to the patient an anti-ErbB3 antibody (e.g., seribantumab), a CDK4/6 inhibitor (e.g., palbociclib), and an endocrine based therapy (e.g., letrozole or fulvestrant).

In another aspect, methods of treating a human patient with a ER+, HER2− breast cancer are provided, the method comprising administering to the patient an anti-ErbB3 antibody (e.g., seribantumab) and an endocrine-based therapy (e.g., letrozole or fulvestrant). In one embodiment, the method does not comprise administration of a CDK4/6 inhibitor (e.g., palbociclib, abemaciclib, or ribociclib). In another embodiment, the method comprises administering to the patient an anti-ErbB3 antibody (e.g., seribantumab) and fulvestrant. In another embodiment, the method comprises administering to the patient an anti-ErbB3 antibody (e.g., seribantumab) and letrozole.

In one embodiment, no more than three other antineoplastic agents (e.g., CDK4/6 inhibitors and/or endocrine based therapies) are administered in combination with seribantumab within a treatment cycle. In another embodiment, no more than two other antineoplastic agents are administered in combination with seribantumab within a treatment cycle. In another embodiment, no more than one other antineoplastic agent is administered in combination with seribantumab within a treatment cycle.

In one embodiment, a treatment cycle is 21 days. In another embodiment, the treatment comprises at least 1, 2, 3, 4, 5, 6, 7, 8, 9, 10 or 11 cycles. Treatment is continued for any suitable period of time (e.g., until a complete response (CR) has been achieved). In one embodiment, the treatment is administered for at least 1 month, 2 months, 3 months, 4 months, 5 months, 6 months, 7 months, 8 months, 9 months, 10 months, or 11 months. In another embodiment, the treatment is administered for at least one year. In another embodiment, the treatment is administered for at least two years.

The therapeutic agents described herein (e.g., seribantumab, palbociclib, letrozole, and fulvestrant) can be administered to a patient by any suitable means. In one embodiment, seribantumab is formulated for intravenous administration. In one embodiment, palbociclib is formulated for oral administration (e.g., as a capsule or tablet). In one embodiment, letrozole is formulated for oral administration (e.g., as a capsule or tablet). In one embodiment, fulvestrant is formulated as a sterile solution for intramuscular injection.

In one embodiment, the dose of the anti-ErbB3 antibody (e.g., seribantumab), the CDK4/6 inhibitor (e.g., palbociclib) and the endocrine based therapy (e.g., letrozole or fulvestrant) is a dose that is fixed irrespective of the weight of the patient. For example, seribantumab may be administered at a fixed dose of 3 g without regard to the patient's weight. Palbociclib may be administered at a fixed dose of a 125 mg capsule without regard to the patient's weight. Letrozole may be administered at a fixed dose of a 2.5 mg without regard to the patient's weight. Fulvestrant may be administered at a fixed dose of 500 mg without regard to the patient's weight. In certain embodiments, dosage regimens are adjusted to provide the optimum desired response (e.g., an effective response).

In one aspect, palbociclib, letrozole, and seribantumab are administered in combination according to a particular dosage regimen. In one embodiment, a 125 mg palbociclib capsule is administered orally once daily for 21 consecutive days, followed by 7 days off treatment for a 28 day cycle. In this embodiment, 2.5 mg of letrozole is given once daily continuously throughout the 28 day cycle. In this embodiment, seribantumab is administered at a dose of 3 g every two weeks by IV infusion throughout the cycle.

In another aspect, palbociclib, fulvestrant, and seribantumab are administered in combination according to a particular dosage regimen. In this embodiment, a 125 mg palbociclib capsule is administered orally once daily for 21 consecutive days, followed by 7 days off treatment for a 28 day cycle. In this embodiment, fulvestrant is administered at a dose of 500 mg on days 1, 15, 29, and once monthly or once every 28 days thereafter. In this embodiment, seribantumab is administered at a dose of 3 g every two weeks by IV infusion throughout the cycle.

Accordingly, in one aspect, methods of treating a human patient with a ER+, HER2− breast cancer are provided, the methods comprising administering to the patient:
I) one palbociclib 125 mg capsule taken orally once daily for 21 consecutive days, followed by 7 days off treatment to comprise a complete cycle of 28 days;
II) either a) or b) wherein a) is letrozole, 2.5 mg given once daily continuously throughout the 28-day cycle, and b) is fulvestrant administered at a dose of 500 mg on days 1, 15, 29, and once monthly or once every 28 days thereafter;
and
   seribantumab at a dose of 3 g every two weeks by IV infusion.

In another aspect, methods of treating a patient who has been previously treated with palbociclib and a hormonal therapy, and whose cancer has progressed on this treatment, are provided, the method comprising concurrently administering to the patient:
I) one palbociclib 125 mg capsule taken orally once daily for 21 consecutive days followed by 7 days off treatment to comprise a complete cycle of 28 days;
II) either a) or b) wherein a) is letrozole, 2.5 mg given once daily continuously throughout the 28-day cycle, and b) is fulvestrant administered at a dose of 500 mg on days 1, 15, 29, and once monthly or once every 28 days thereafter and wherein if the patient previously was treated with fulvestrant, then the patent is administered a) and if the patient was previously treated with letrozole, then the patent is administered b); and
   seribantumab at a dose of 3 g every two weeks by IV infusion.

In another aspect, methods of treating a patient with ER/PR+, HER2− breast cancer expressing HRG as measured by RNA in-situ hybridization (RNA-ISH) are provided. In one embodiment, the breast cancer is locally advanced or metastatic breast cancer. In another embodiment, the method comprises a 28-day cycle, wherein:
I) seribantumab is administered at a dose of 3000 mg intravenously (IV) on days 1 and 15 of the cycle, and
II) fulvestrant is administered at a dose of 500 mg intramuscularly (IM) on days 1 and 15 of the cycle.

In one embodiment, the method comprises at least one subsequent treatment cycle. In another embodiment, fulvestrant is administered only on day 1 of each subsequent treatment cycle.

In another aspect, the method of treating a patient with ER/PR+, HER2− breast cancer expressing HRG as measured by RNA in-situ hybridization (RNA-ISH) comprises a 28-day cycle, wherein:
I) seribantumab is administered at a dose of 3000 mg IV on days 1 and 15 of the cycle, and
II) letrozole is administered at a dose of 2.5 mg orally once per day during the cycle.

In one embodiment, a heregulin RNA in situ hybridization (RNA-ISH) score of 1+ of higher has been measured in a biological sample from the patient prior to treatment.

The efficacy of the treatment methods provided herein can be assessed using any suitable means. In one embodiment, the treatment produces at least one therapeutic effect selected from the group consisting of reduction in size of a tumor, reduction in metastasis, complete remission, partial remission, stable disease, increase in overall response rate, or a pathologic complete response. In one embodiment, the treatment results in the patient exhibiting stable disease, a partial response, or a complete response.

Further provided are kits that include an anti-ErbB3 antibody, such as seribantumab, a CDK4/6 inhibitor, such as palbociclib, and an endocrine based therapy, such as letrozole or fulvestrant. In one embodiment, the kit comprises: (a) a dose of seribantumab, (b) a dose of palbociclib, (c) a dose of letrozole or fulvestrant, and (d) instructions for using letrozole or fulvestrant in combination with seribantumab and palbociclib, in the methods described herein. In another embodiment, the kit comprises: (a) a dose of seribantumab, (b) a dose of letrozole or fulvestrant, and (c) instructions for using letrozole or fulvestrant in combination with seribantumab, in the methods described herein.

### DETAILED DESCRIPTION

### I. Definitions

As used herein, the term “subject” or “patient” is a human patient (e.g., a patient having ER+, HER2− HRG+ metastatic breast cancer).

As used herein, the term “estrogen receptor positive” (ER+) refers to tumors (e.g., carcinomas), typically breast tumors, in which the tumor cells score positive (i.e., using conventional histopathology methods) for estrogen receptor (ER). According to recommendations provided by the College of American Pathologists (CAP) and the American Society of Clinical Oncology (ASCO), a tumor is ER+ if at least 1% of the tumor cells tested (e.g., by immunohistochemistry) score ER positive.

The terms “ErbB2,” “HER2,” and “HER2 receptor,” as used interchangeably herein, refer to the protein product of the human neu oncogene, also referred to as the ErbB2 oncogene or the HER2 oncogene. According to guidelines provided by CAP and the ASCO, a tumor designated HER2 negative (HER2-) is a tumor in which an immunoassay such as immunohistochemistry (IHC) test shows no staining or membrane staining in <30% of tumor cells. For one such assay, marketed as HERCEPTEST®, a score of 0 or 1+ is considered HER2 negative, a score of 2+ is considered equivocal—requiring further testing by fluorescence in-situ hybridization (FISH) for definitive characterization, and a score of 3+ is considered HER2 positive. Therefore a patient with a biopsy scoring 0 or 1+ by HERCEPTEST, or 2+ by HERCEPTEST and negative by FISH is considered HER2 negative, while a patient scoring 3+ by HERCEPTEST or 2+ by HERCEPTEST and FISH positive is deemed HER2 positive.

As used herein, “HRG” indicates any and all isotypes of heregulin (neuregulin-1, “NRG”), a set of naturally occurring ligands of ErbB3. HRG expression can be evaluated, for example, using a RNA in situ-hybridization (ISH)-based assay, e.g., according to the protocol described in Example 1 of U.S. Ser. No. 14/965,301; WO 2015/100459, which is expressly incorporated herein by reference. In one embodiment, the RNA-ISH is read out via a chromogenic signal. In a particular embodiment, the probes used to detect HRG by RNA-ISH hybridize specifically to a nucleic acid that comprises nucleotides 442-2977 of the nucleotide sequence set forth in GenBank accession number NM-013956 (SEQ ID NO:13). In certain embodiments the probes hybridize specifically to RNAs encoding each of the HRG isoforms α, β1, β1b, β1c, β1d, β2, β2b, β3, β3b, γ, γ2, γ3, ndf43, ndf34b, and GGF2. In another embodiment, the HRG score is determined by RT-PCR using probes specific for HRG.

The terms “ErbB3” and “HER3,” as used interchangeably herein, refer to human ErbB3 protein, as described in U.S. Pat. No. 5,480,968. The human ErbB3 protein sequence is shown in SEQ ID NO:4 of U.S. Pat. No. 5,480,968, wherein the first 19 amino acids (aas) correspond to the leader sequence that is cleaved from the mature protein. ErbB3 is a member of the ErbB family of receptors, other members of which include ErbB1 (EGFR), ErbB2 (HER2/Neu) and ErbB4. ErbB3 itself lacks tyrosine kinase activity, but is itself phosphorylated upon dimerization of ErbB3 with another ErbB family receptor, e.g., ErbB1 (EGFR), ErbB2 and ErbB4, which are receptor tyrosine kinases. Ligands for the ErbB family receptors include heregulin (HRG), betacellulin (BTC), epidermal growth factor (EGF), heparin-binding epidermal growth factor (HB-EGF), transforming growth factor alpha (TGF-α), amphiregulin (AR), epigen (EPG) and epiregulin (EPR). The amino acid sequence of human ErbB3 is provided at Genbank Accession No. NP_001973.2 (receptor tyrosine-protein kinase erbB-3 isoform 1 precursor) and is assigned Gene ID: 2065.

As used herein, the term “ErbB3 inhibitor” is intended to include therapeutic agents that inhibit, downmodulate, suppress or downregulate activity of ErbB3. The term is intended to include chemical compounds, such as small molecule inhibitors, and biologic agents, such as antibodies, interfering RNA (shRNA, siRNA), soluble receptors and the like. An exemplary ErbB3 inhibitor is an anti-ErbB3 antibody, such as seribantumab.

As used herein, the term “agent,” refers to an active molecule, e.g., a therapeutic protein, e.g., a drug.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. Effective treatment may refer to alleviation of at least one symptom of cancer.

As used herein, the term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. An effective amount can be administered in one or more administrations.

As used herein, the term “administer” or “administration” refers to the act of injecting or otherwise physically delivering a substance as it exists outside the body (e.g., a formulation of the molecules disclosed herein) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent.

The terms “treat,” “treating,” and “treatment,” as used herein, refer to therapeutic or preventative measures described herein. The methods of “treatment” employ administration to a subject, the combination disclosed herein in order to prevent, cure, delay, reduce the severity of, or ameliorate one or more symptoms of the disease or disorder or recurring disease or disorder, or in order to prolong the survival of a subject beyond that expected in the absence of such treatment.

As used herein, adjunctive or combined administration (coadministration) includes simultaneous administration of the agents in the same or different dosage form, or separate administration of the agents (e.g., sequential administration). For example, the agents can be formulated for separate administration and administered concurrently or sequentially. Such concurrent or sequential administration preferably results in the agents being simultaneously present in treated patients.

### II. Anti-ErbB3 Antibodies

Anti-ErB3 antibodies (or VH/VL domains derived therefrom) suitable for use in the invention can be generated using methods well known in the art. Alternatively, art recognized anti-ErbB3 antibodies can be used, for example, AV-203 (as described in U.S. Pat. No. 8,481,687), GSK2849330 (as described in U.S. Pat. No. 9,085,622), KTN3379 (as described in U.S. Pat. No. 9,220,775), duligotuzumab (as described in U.S. Pat. No. 8,597,652), elgemtumab (as described in U.S. Pat. No. 8,735,551), futuximab (as described in WO2008/104183), lumretuzumab (as described in U.S. Pat. No. 8,859,737), and patritumab (as described in U.S. Pat. No. 7,705,130). Antibodies that compete with any of these art-recognized antibodies for binding to ErbB3 also can be used.

An exemplary anti-ErbB3 antibody is seribantumab (also known as “MM-121” or “Ab #6”) or antigen binding fragments and variants thereof. Seribantumab is a human monoclonal anti-ErbB3 IgG2 (see, e.g., U.S. Pat. Nos. 7,846,440; 8,691,771 and 8,961,966; 8,895,001, U.S. Patent Publication Nos., 20110027291, 20140127238, 20140134170, and 20140248280), as well as international publication Nos. WO/2013/023043, WO/2013/138371, WO/2012/103341, and U.S. Provisional Patent Application Ser. No. 62/090,780, the teachings of which are expressly incorporated herein by reference).

In one embodiment, the anti-ErbB3 antibody comprises the heavy and light chain CDRs or variable regions of seribantumab. Accordingly, in one embodiment, the antibody comprises the CDR1, CDR2, and CDR3 domains of the VH region of seribantumab having the sequence set forth in SEQ ID NO: 10 and the CDR1, CDR2 and CDR3 domains of the VL region of seribantumab having the sequence set forth in SEQ ID NO: 12. In another embodiment, the antibody comprises heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. In another embodiment, the antibody comprises VH and/or VL regions having the amino acid sequences set forth in SEQ ID NO: 10 and SEQ ID NO: 12, respectively. In another embodiment, the anti-ErbB3 antibody comprises VH and/or VL regions encoded by the nucleic acid sequences set forth in SEQ ID NOs: 9 and 11, respectively. In another embodiment, the anti-ErbB3 antibody comprises heavy and/or light chains having the amino acid sequences set forth in SEQ ID NO: 7 and SEQ ID NO: 8, respectively. In another embodiment, an antibody is used that competes for binding with and/or binds to the same epitope on human ErbB3 as the above-mentioned antibodies. In a particular embodiment, the epitope comprises residues 92-104 of human ErbB3 (SEQ ID NO: 14). In another embodiment, the antibody competes with seribantumab for binding to human ErbB3 and has at least 90% variable region amino acid sequence identity with the above-mentioned anti-ErbB3 antibodies (see, e.g., U.S. Pat. No. 7,846,440 and US Patent Publication No. 20100266584).

### III. CDK4/6 Inhibitor

Art recognized CDK4/6 inhibitors can be used. An exemplary CDK4/6 inhibitor is palbociclib. Palbociclib (codenamed PD-0332991, trade name IBRANCE) is a drug for the treatment of ER-positive and HER2-negative breast cancer. It is a selective inhibitor of the cyclin-dependent kinases CDK4 and CDK6. IBRANCE capsules for oral administration contain 125 mg, 100 mg, or 75 mg of palbociclib, a kinase inhibitor. The molecular formula for palbociclib is C₂₄H₂₉N₇O₂. The molecular weight is 447.54 daltons. The chemical name is 6-acetyl-8-cyclopentyl-5-methyl-2-{[5-(piperazin-1-yl)pyridin-2-yl]amino}pyrido[2,3-d]pyrimidin-7(8H)-one, and its structural formula is:

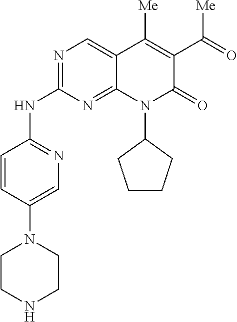 [SMILES: CC(=O)c1c(C)c2cnc(Nc3ccc(N4CCNCC4)cn3)nc2n(C2CCCC2)c1=O]

Palbociclib is a yellow to orange powder with pKa of 7.4 (the secondary piperazine nitrogen) and 3.9 (the pyridine nitrogen). At or below pH 4, palbociclib behaves as a high-solubility compound. Above pH 4, the solubility of the drug substance reduces significantly. Palbociclib contains the following inactive ingredients: Microcrystalline cellulose, lactose monohydrate, sodium starch glycolate, colloidal silicon dioxide, magnesium stearate, and hard gelatin capsule shells. The light orange, light orange/caramel and caramel opaque capsule shells contain gelatin, red iron oxide, yellow iron oxide, and titanium dioxide; and the printing ink contains shellac, titanium dioxide, ammonium hydroxide, propylene glycol and simethicone.

The recommended dose of palbociclib is a 125 mg capsule taken orally once daily for 21 consecutive days followed by 7 days off treatment to comprise a complete cycle of 28 days. IBRANCE should be taken with food.

When coadministered with palbociclib, the recommended dose of letrozole is 2.5 mg taken once daily continuously throughout the 28-day cycle.

When coadministered with palbociclib, the recommended dose of fulvestrant is 500 mg administered on Days 1, 15, 29, and once monthly thereafter.

Another exemplary CDK4/6 inhibitor is abemaciclib. Abemaciclib (codenamed LY2835219; trade name IBRANCE) is an investigational drug for various types of cancer. It is an orally selective inhibitor of the cyclin-dependent kinases CDK4 and CDK6. The molecular formula for abemaciclib is C₂₇H₃₂F₂N₈. The molecular weight is 506.61 daltons. The chemical name is 2-pyrimidinamine, N-(5-((4-ethyl-1-piperazinyl)methyl)-2-pyridinyl)-5-fluoro-4-(4-fluoro-2-methyl-1-(1-methylethyl)-1H-benzimidazol-6-yl), and its structural formula is:

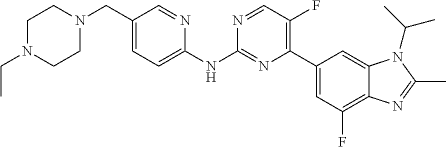 [SMILES: CCN1CCN(Cc2ccc(Nc3ncc(F)c(-c4cc(F)c5nc(C)n(C(C)C)c5c4)n3)nc2)CC1]

Another exemplary CDK4/6 inhibitor is ribociclib. Ribociclib (codenamed LEE011; trade name KISQUALI) is a drug for the treatment of various cancers, including hormone receptor-positive and HER2-negative advanced or metastatic breast cancer. It is an orally available, highly selective inhibitor of the cyclin-dependent kinases CDK4 and CDK6. The molecular formula for Ribociclib is C₂₃H₃₀N₈O. The molecular weight is 434.55 daltons. The chemical name is 7-cyclopentyl-N,N-dimethyl-2-((5-(piperazin-1-yl)pyridin-2-yl)amino)-7H-pyrrolo[2,3-d]pyrimidine-6-carboxamide, and its structural formula is:

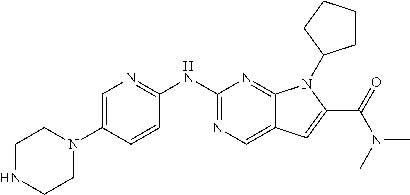 [SMILES: CN(C)C(=O)c1cc2cnc(Nc3ccc(N4CCNCC4)cn3)nc2n1C1CCCC1]

KISQALI tablets are recommended to be are taken daily with or without food. Recommended starting dose: 600 mg orally (three 200 mg tablets) taken once daily with or without food for 21 consecutive days followed by 7 days off treatment.

### IV. Endocrine Based Therapy

Art recognized endocrine based therapies can be used. Exemplary endocrine based therapies include non-steroidal aromatase inhibitors (e.g., letrozole, anostrozole) and selective estrogen receptor degraders (e.g., fulvestrant, brilanestrant, elacestrant).

An exemplary endocrine based therapy is letrozole. Letrozole (trade name FEMARA) is a nonsteroidal aromatase inhibitor (inhibitor of estrogen synthesis). Letrozole inhibits the aromatase enzyme by competitively binding to the heme of the cytochrome P450 subunit of the enzyme, resulting in a reduction of estrogen biosynthesis in all tissues. It is chemically described as 4,4′-(1H-1,2,4-Triazol-1-ylmethylene)dibenzonitrile, and its structural formula is

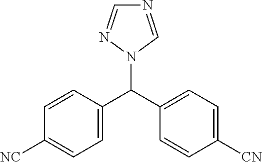 [SMILES: N#Cc1ccc(C(c2ccc(C#N)cc2)n2cncn2)cc1]

Letrozole is a white to yellowish crystalline powder, practically odorless, freely soluble in dichloromethane, slightly soluble in ethanol, and practically insoluble in water. It has a molecular weight of 285.31, empirical formula C₁₇H₁₁N₅, and a melting range of 184° C.−185° C.

FEMARA (letrozole tablets) is available as 2.5 mg tablets for oral administration. Letrozole contains the following inactive Ingredients: colloidal silicon dioxide, ferric oxide, hydroxypropyl methylcellulose, lactose monohydrate, magnesium stearate, maize starch, microcrystalline cellulose, polyethylene glycol, sodium starch glycolate, talc, and titanium dioxide.

The recommended dose of FEMARA (letrozole tablets) is one 2.5 mg tablet administered once a day, without regard to meals.

Another exemplary endocrine based therapy is anastrozole (trade name ARIMIDEX). ARIMIDEX (anastrozole) is an orally available aromatase inhibitor which competively blocks the conversion of androgens to estrogens in peripheral (extra-gonadal) tissues. The chemical name is a,a,a′,a′-Tetramethyl-5-(1H-1,2,4-triazol-1-ylmethyl)-1,3-benzenediacetonitrile. The molecular formula is C₁₇H₁₉N₅ and its structural formula is:

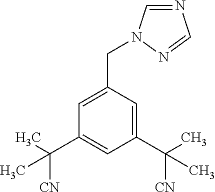

Anastrozole is freely soluble in methanol, acetone, ethanol, and tetrahydrofuran, and very soluble in acetonitrile. Each tablet contains as inactive ingredients: lactose, magnesium stearate, hydroxypropylmethylcellulose, polyethylene glycol, povidone, sodium starch glycolate, and titanium dioxide. ARIMIDEX is available as 1 mg tablets for oral administration and the recommended dose of ARIMIDEX is one tablet daily.

Another exemplary endocrine based therapy is fulvestrant (trade name FASLODEX). FASLODEX (fulvestrant) Injection for intramuscular administration is an estrogen receptor antagonist. The chemical name is 7-alpha-[9-(4,4,5,5,5-penta fluoropentylsulphinyl) nonyl]estra-1,3,5-(10)-triene-3,17beta-diol. The molecular formula is C₃₂H₄₇F₅O₃S and its structural formula is:

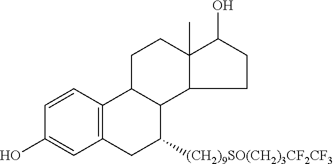

Fulvestrant is a white powder with a molecular weight of 606.77. The solution for injection is a clear, colorless to yellow, viscous liquid. Each injection contains as inactive ingredients: 10% w/v Alcohol, USP, 10% w/v Benzyl Alcohol, NF, and 15% w/v Benzyl Benzoate, USP, as co-solvents, and made up to 100% w/v with Castor Oil, USP as a co-solvent and release rate modifier.

The recommended dose of FASLODEX is 500 mg and should be administered intramuscularly into the buttocks slowly (1-2 minutes per injection) as two 5 mL injections, one in each buttock, on days 1, 15, 29 and once monthly thereafter. A dose of 250 mg is recommended in patients with moderate hepatic impairment to be administered intramuscularly into the buttock slowly (1-2 minutes) as one 5 mL injection on days 1, 15, 29 and once monthly thereafter.

Another exemplary endocrine based therapy is brilanestrant (Code names: GDC-0810, ARN-810, RG-6046, RO-7056118). Brilanestrant is an investigational drug for the treatment of metastatic estrogen receptor-positive breast cancer. It is a non-steroidal combined selective estrogen receptor modulator (SERM) and selective estrogen receptor degrader (SERD). The chemical name is (2E)-3-{4-[(1E)-2-(2-Chloro-4-fluorophenyl)-1-(1H-indazol-5-yl)but-1-en-1-yl]phenyl}prop-2-enoic acid. The molecular formula is C₂₆H₂₀ClFN₂O₂ and its structural formula is:

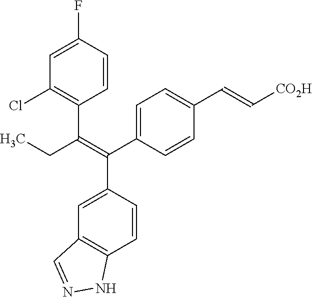 [SMILES: CC/C(=C(/c1ccc(/C=C/C(=O)O)cc1)c1ccc2[nH]ncc2c1)c1ccc(F)cc1Cl]

Brilanestrant is orally available and does not need to be administered by intramuscular injection.

Another exemplary endocrine based therapy is elacestrant (Code names: RAD-1901, ER-306323). Elacestrant is an investigational drug for the treatment of estrogen receptor-positive breast cancer, endometrial cancer, and kidney cancer. It is a non-steroidal combined selective estrogen receptor modulator (SERM) and selective estrogen receptor degrader (SERD). The chemical name is (6R)-6-{2-[ethyl({4-[2-(ethylamino)ethyl]phenyl}methyl)amino]-4-methoxyphenyl}-5,6,7,8-tetrahydronaphthalen-2-ol. The molecular formula is C₃₀H₃₈N₂O₂ and its structural formula is:

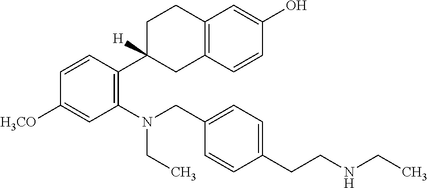 [SMILES: CCNCCc1ccc(CN(CC)c2cc(OC)ccc2[C@@H]2CCc3cc(O)ccc3C2)cc1]

Elacestrant is orally available and does not need to be administered by intramuscular injection.

### V. Outcomes

Provided herein are compositions and methods for treating ER+, HER2− breast cancer (e.g., metastatic ER+, HER2− breast cancer) in a human patient, comprising administering to the patient an anti-ErbB3 antibody (e.g., seribantumab or istiratumab), a CDK4/6 inhibitor (e.g., palbociclib, abemaciclib, or ribociclib), and an endocrine based therapy (e.g., letrozole or fulvestrant) according to a particular clinical dosage regimen (i.e., at a particular dose amount and according to a specific dosing schedule). Also provided herein, are composition and methods for treating ER+, HER2− breast cancer (e.g., metastatic ER+, HER2− breast cancer) in a human patient, comprising administering to the patient an anti-ErbB3 antibody (e.g., seribantumab or istiratumab), and an endocrine based therapy (e.g., letrozole or fulvestrant) according to a particular clinical dosage regimen (i.e., at a particular dose amount and according to a specific dosing schedule)

Treatment outcomes can be evaluated using standard measures for tumor response. Target lesion (tumor) responses to therapy are classified as:
Complete Response (CR): Disappearance of all target lesions. Any pathological lymph nodes (whether target or non-target) must have reduction in short axis to <10 mm;
Partial Response (PR): At least a 30% decrease in the sum of the diameters of target lesions, taking as reference the baseline sum diameters;
Progressive Disease (PD: At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression); and
Stable Disease (SD): Neither sufficient shrinkage to qualify for PR nor sufficient increase to qualify for PD, taking as reference the smallest sum diameters while on study. (Note: a change of 20% or less that does not increase the sum of the diameters by 5 mm or more is coded as stable disease). To be assigned a status of stable disease, measurements must have met the stable disease criteria at least once after study entry at a minimum interval of 6 weeks.

Non-target lesion responses to therapy are classified as:
Complete Response (CR): Disappearance of all non-target lesions and normalization of tumor marker levels. All lymph nodes must be non-pathological in size (<10 mm short axis). If tumor markers are initially above the upper normal limit, they must normalize for a patient to be considered in complete clinical response;
Non-CR/Non-PD: Persistence of one or more non-target lesion(s) and/or maintenance of tumor marker level above the normal limits; and
Progressive Disease (PD): Either or both of appearance of one or more new lesions and unequivocal progression of existing non-target lesions. In this context, unequivocal progression must be representative of overall disease status change, not a single lesion increase. Patients treated according to the methods disclosed herein preferably experience improvement in at least one sign of cancer. For example, the treatment may produce at least one therapeutic effect selected from the group consisting of reduction in size of a tumor, reduction in metastasis, complete remission, partial remission, stable disease, increase in overall response rate, or a pathologic complete response. Response may also be measured by a reduction in the quantity and/or size of measurable tumor lesions. Measurable lesions are defined as those that can be accurately measured in at least one dimension (longest diameter is to be recorded) as >10 mm by CT scan (CT scan slice thickness no greater than 5 mm), 10 mm caliper measurement by clinical exam or >20 mm by chest X-ray. The size of non-target lesions, e.g., pathological lymph nodes can also be measured for improvement. Lesions can be measured using, e.g., x-ray, CT, or MRI images. Microscopy, cytology or histology can be also used to evaluate responsiveness to a therapy. An effusion that appears or worsens during treatment when a measurable tumor has otherwise met criteria for response or stable disease can be considered to indicate tumor progression, but only if there is cytological confirmation of the neoplastic origin of the effusion.

In another embodiment, the patient so treated experiences tumor shrinkage and/or decrease in growth rate, i.e., suppression of tumor growth. In another embodiment, tumor cell proliferation is reduced or inhibited. Alternately, one or more of the following can indicate a beneficial response to treatment: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent. Other indications of a favorable response include reduction in the quantity and/or size of measurable tumor lesions or of non-target lesions.

### VI. Kits and Unit Dosage Forms

Also provided herein are kits which include an anti-ErbB3 antibody (e.g., seribantumab or istiratumab), a CDK4/6 inhibitor (e.g., palbociclib, abemaciclib, or ribociclib) and an endocrine based therapy (e.g., letrozole or fulvestrant), in a therapeutically effective amount adapted for use in the preceding methods. In another embodiment, the kits include an anti-ErbB3 antibody (e.g., seribantumab or istiratumab) and an endocrine based therapy (e.g., letrozole or fulvestrant), in a therapeutically effective amount adapted for use in the preceding methods. The kits optionally also can include instructions, e.g., comprising administration schedules, to allow a practitioner (e.g., a physician, nurse, or patient) to administer the therapeutic agents contained therein to a patient having cancer. The kit also can include a syringe. Instruments or devices necessary for administering the pharmaceutical composition(s) also may be included in the kits.

In one embodiment, the present invention provides a kit comprising: (a) a dose of seribantumab or istiratumab, (b) a dose of palbociclib, (c) a dose of letrozole or fulvestrant, and (d) instructions for using letrozole or fulvestrant in combination with seribantumab or istiratumab and palbociclib, in the methods described herein. In another embodiment, the kit comprises (a) a dose of seribantumab or istiratumab, (b) a dose of letrozole or fulvestrant, and (c) instructions for using letrozole or fulvestrant in combination with seribantumab or istiratumab, in the methods described herein.

The following examples are merely illustrative and should not be construed as limiting the scope of this disclosure in any way as many variations and equivalents will become apparent to those skilled in the art upon reading the present disclosure.

The contents of all references, Genbank entries, patents and published patent applications cited throughout this application are expressly incorporated herein by reference.

### EXAMPLES

### Materials & Methods

### Cell Lines, Cell Culture, and Reagents

MCF-7, T47D, ZR75-1 and HCC-1428 and were obtained from the American Type Culture Collection (“ATCC” Rockville, Md., USA). All cells were cultured in RPMI1640 medium supplemented with 10% v/v Heat inactivated FBS, 5% v/v L-glutamine and 5% v/v penicillin-streptomycin solution. All culture reagents were from Gibco unless otherwise stated. When hormone-free conditions were required, cells were cultured in phenol red free RPMI1640 medium supplemented with 10% v/v charcoal stripped, heat inactivated FBS, 5% v/v L-glutamine and 5% v/v penicillin-streptomycin solution. To ensure low growth factor conditions for growth factor stimulation assays, cells were cultured in low serum conditions such as 3% v/v heat inactivated FBS or 1% v/v heat inactivated FBS with normal supplementation of other media components. All cell lines were cultured at 37° C. in a humid atmosphere with 95% air, 5% CO₂. The identities of all cells used were verified by microsatellite analyses at ATCC. Recombinant heregulin (HRGβ1) was from R&D Systems (396-HB). Cell Titer-Glo assay reagents were from Promega. Estradiol (E8875) and fulvestrant (14409) were from Sigma-Aldrich. Tamoxifen (S1972), palbociclib (S1579), abemaciclib (S7158) and ribociclib (S7440) were all from SelleckChem.

### Proliferation Assays

Cells were seeded in duplicate or triplicate at 1500 to 5000 cells per well in 96 well plates, Opaque-walled 96-well plates with clear bottom: 96-well, Black/Clear, Flat bottom with lid, FALCON, 353219 in reduced serum conditions at either 3% v/v FBS or 1% v/v FBS. The day after plating recombinant HRGβ1 was added to yield a final concentration of 10 nM. Control wells received media without HRGβ1. Plates were then incubated for the indicated time period of 4 to 6 days at 37° C. in a humid atmosphere with 95% air, 5% CO₂.

For studies including HRGβ1 and the ErbB3 targeting mAb, seribantumab (MM-121) or CDK inhibitors such as palbociclib, abemaciclib or ribociclib, cells were seeded in duplicate or triplicate at 1500 to 5000 cells per well in 96 well plates, Opaque-walled 96-well plates with clear bottom: Nano-Culture plates, MS pattern, low binding, SCIVAX Life Science NCP-LS96-10 in reduced serum conditions at either 3% v/v FBS or 1% v/v FBS. The day after plating recombinant HRGβ1 was added to yield a final concentration of 10 nM. Control wells received media without HRGβ1. Where indicated seribantumab was added to achieve a final concentration of 1 μM or over a dilution series to achieve 10 dilutions per plate to achieve a dose-response curve with 2 controls wells per row. Where indicated CDK inhibitors were added at 10 μM and dilutions were performed to generate a dose-response curve with 2 controls wells per row. Plates were then incubated for the indicated time period of 4 to 6 days at 37° C. in a humid atmosphere with 95% air, 5% CO₂. Growth inhibition was calculated as a function of the relative inhibition or proliferation of cells treated with either growth factor or antagonists to cells treated with diluent only, over the same time period.

For studies including HRGβ1 and the ErbB3 targeting mAb, seribantumab (MM-121) or CDK inhibitors such as palbociclib, abemaciclib or ribociclib cells were seeded in duplicate or triplicate at 1500 to 5000 cells per well in 96 well plates, Opaque-walled 96-well plates with clear bottom: Nano-Culture plates, MS pattern, low binding, SCIVAX Life Science NCP-LS96-10 in reduced serum conditions at either 3% v/v FBS or 1% v/v FBS. The day after plating recombinant HRGβ1 was added to yield a final concentration of 10 nM. Control wells received media without HRGβ1. Where indicated, seribantumab was added to achieve a final concentration of 1 μM or over a dilution series to achieve 10 dilutions per plate to achieve a dose-response curve with 2 controls wells per row. Where indicated CDK inhibitors were added at 10 μM and dilutions were performed to generate a dose-response curve with 2 controls wells per row. Plates were then incubated for the indicated time period of 4 to 6 days at 37° C. in a humid atmosphere with 95% air, 5% CO₂.

To measure proliferation, Cell Titer-Glo (CTG) assays were performed as per manufacturer's instructions. Specifically, reagent-1 and reagent-2 were equilibrated to room temperature at which point reagent-1 was added to reagent-2 and mixed by vortex. Test plates containing cells were equilibrated to room temperature for 30 minutes at which point an equal volume of CTG reagent was added to each well of the test plate, typically 100 μl to give a final volume of 200 μl per well. Each plate was then sealed with a foil plate sealer and mixed on an orbital shaker for 10 minutes to lyse cells and release cellular ATP. Following mixing plates were incubated at room temperature for 15 minutes to stabilize the luminescent signal. Data was collected by measuring relative luminescence on a Synergy H₁ plate reader using the luminescence program. Growth was calculated and expressed as a relative value to the unstimulated control wells on each individual plate. Growth inhibition was calculated as a function of the relative inhibition of cells treated with antagonists to the growth of cells treated with diluent only, over the same time period. Data was then plotted using GraphPad Prism software.

### Example 1: Phenotypically Distinct HRG Positive Cancer Cells Impacts Standard of Care Therapies in Metastatic Breast Cancer Models

ErbB3 is a member of the human epidermal growth factor receptor (ErbB or HER) family which is comprised of four receptors (ErbB1-4). A defining feature of the ErbB network is that two members of the family, ErbB2 and ErbB3, are non-autonomous. ErbB2 lacks the capacity to interact with a growth-factor ligand, whereas the kinase activity of ErbB3 is defective. Heregulin (HRG), the ErbB3 ligand, has been identified as a potent driver of proliferation and enhanced survival. HRG expression leads to a distinct tumor cell phenotype characterized by an inability to respond to the effects of numerous Standard of Care (SOC) therapies, including chemotherapies, anti-hormonal agents and other targeted therapeutics.

In surveys of HRG expression, HRG+ cells are present in approximately 50% of the cases of most solid tumor types. It is hypothesized that these HRG+ cells are protected from the effects of SOC therapy and continue to proliferate even in the presence of SOC, resulting in limited clinical benefit. In this model, if HRG activity is blocked, HRG+ cells become susceptible to SOC, resulting in enhanced clinical benefit. Seribantumab is a fully human anti-ErbB3 monoclonal antibody designed to block HRG activity by inhibiting the binding of HRG to ErbB3. In the presence of seribantumab, HRG+ tumor cells are predicted to be able to respond to co-administered SOC therapy.

For hormone receptor positive (HR+) breast cancer, hormone deprivation strategies have proven clinical benefit in the adjuvant and metastatic settings. Unfortunately, clinical benefit from these therapies can be short-lived in some patients. Optimal clinical management of these patients requires a comprehensive molecular understanding of the drivers of rapid clinical progression. It has been shown that HRG mRNA expression measured in tumor samples defines a subgroup of patients who derive only limited clinical benefit from SOC when compared to patients whose tumors do not express HRG. This was observed in a previously published Phase 2 clinical study with exemestane, and preclinically with multiple classes of anti-hormonal agents, including letrozole and fulvestrant—treatments that currently represent the mainstay of treatment options for HR+, HER2 negative (HER2−) advanced breast cancer.

The data supports the hypothesis that phenotypically distinct HRG+ cells in breast cancer models persist despite treatment with SOC and various novel classes of therapy. Moreover, the data suggests that addition of seribantumab to these other therapies is important for sustained treatment responses. Continued expansion of HRG+ cells could be the key to rapid clinical progression in breast cancer patients treated with SOC therapy. These findings support the development of seribantumab in combination with anti-hormonal agents in a Phase 3 clinical trial in HR+, HER2− advanced breast cancer.

### Example 2: Heregulin mRNA is Prevalent in Patients with ER+, HER2− Breast Cancer

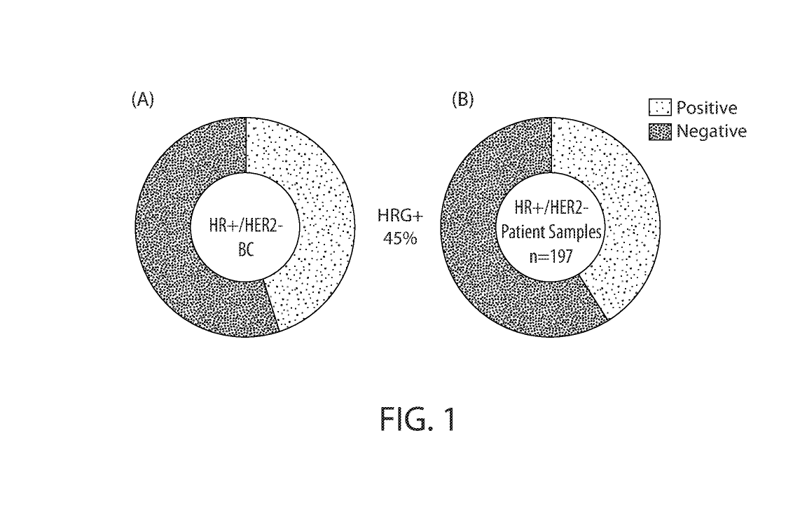

HRG expression in breast cancer cells can contribute to cancer progression and resistance to therapies by activating HER3 signaling. To elaborate on this finding, the prevalence of HRG mRNA in the TCGA public data base and by directly measuring HRG mRNA in 197 ER-positive, HER2-negative breast cancer tumors using a clinically relevant HRG RNA-ISH assay was examined. Both the TCGA database and the patient samples were found to have a prevalence of 45% for HRG mRNA (FIG. 1).

### Example 3: Heregulin Induces Proliferation of ER+, HER2− Breast Cancer Cell Lines

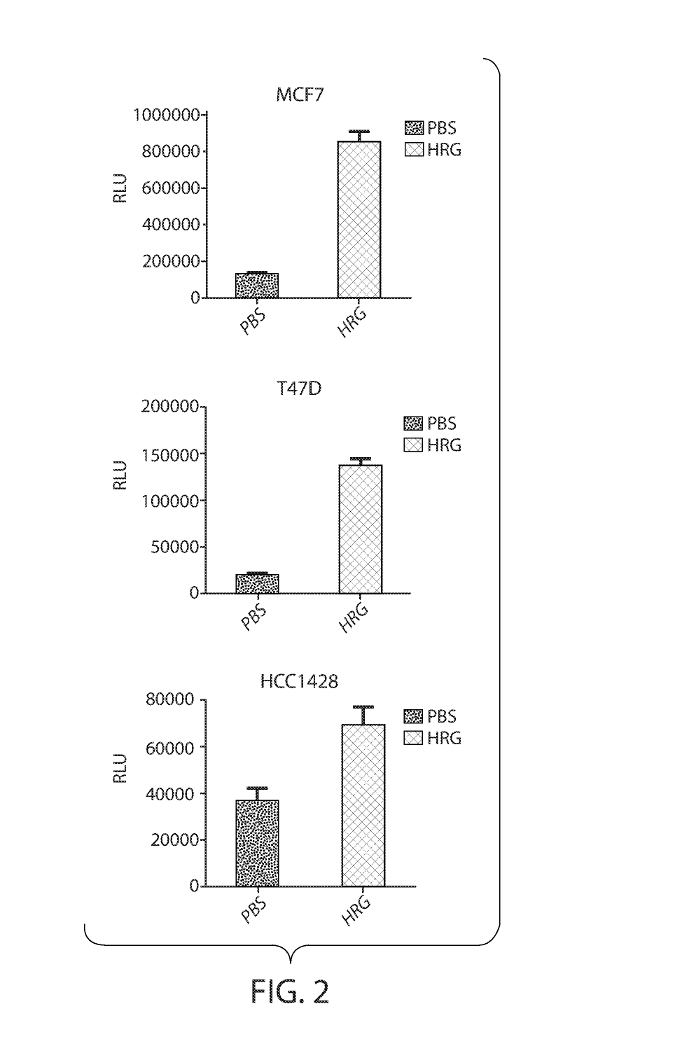

ER+, HER2− breast cancers cell lines with HRG for 6 days were stimulated and proliferation was measured in vitro. Results (FIG. 2) indicate that HRG induced proliferation of MCF7, T47D and HCC1428 cell lines, all of which are ER+, HER2− cellular models. These data support the conclusion that the presence of HRG drives cancer cell proliferation.

### Example 4: Heregulin Augments the Activity of Anti-Hormonal Agents in ER+, HER2− Breast Cancer Cell Lines

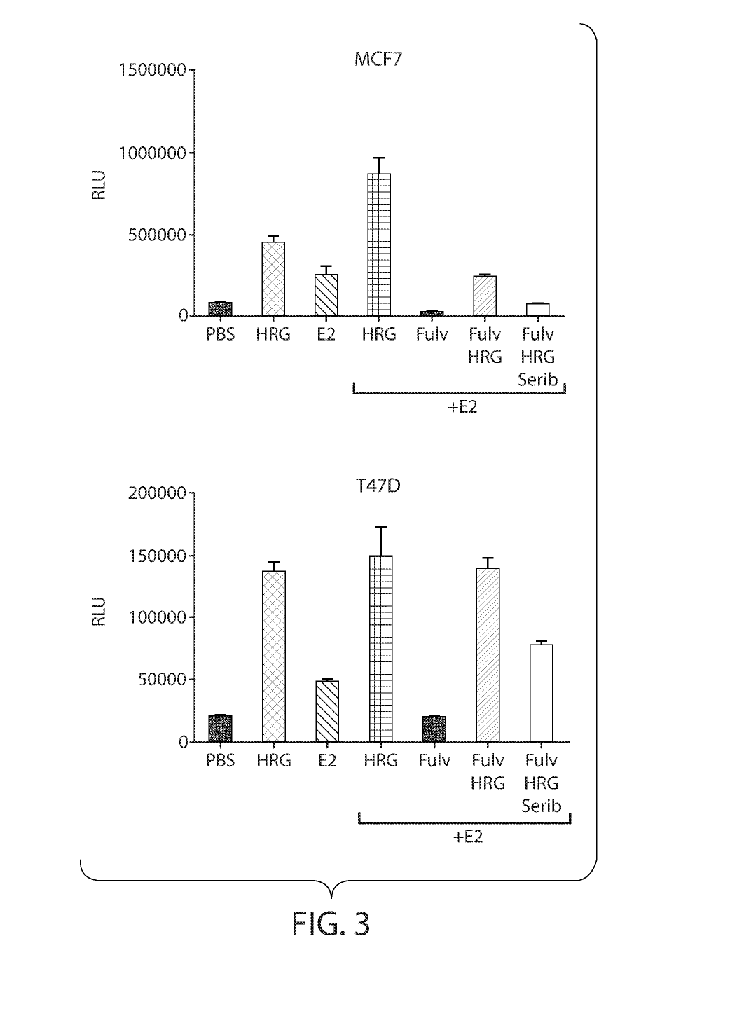

Fulvestrant is classified a “SERD”, selective estrogen receptor degrader and is widely used to treat patients with advanced ER+ breast cancers. SERDs antagonize hormone binding to the receptor and promote degradation of receptor protein, thereby having a dual mechanism of action (MOA) to inhibit hormone receptor signaling and cancer cell growth. As shown in FIG. 3, HRG significantly increased the proliferation of MCF7 and T47D cells, more so than estradiol (E2). In addition, estradiol and HRG in combination resulted in increased proliferation in both cell lines. Fulvestrant (100 nM) was effective at inhibiting estradiol induced proliferation in both MCF7 and T47D cell lines. However, when HRG was present, in addition to estradiol, fulvestrant activity was significantly decreased (FIG. 3). Finally, the addition of seribantumab to cells treated with fulvestrant, estradiol and HRG resulted in restoration of the activity of fulvestrant with the greatest degree of inhibition observed in MCF7 cells. These data indicate that the ErbB3 ligand HRG, which is prevalent in human ER+, HER2− breast cancer, can induce proliferation of breast cancer cell lines and can inhibit the effectiveness of anti-hormonal therapy such as fulvestrant. Further, seribantumab can restore fulvestrant sensitivity in HRG-mediated fulvestrant-resistant cells.

### Example 5: HRG Inhibits the Activity of CDK Inhibitors in ER+, HER2− Breast Cancer Cell Lines and Seribantumab Restores Sensitivity

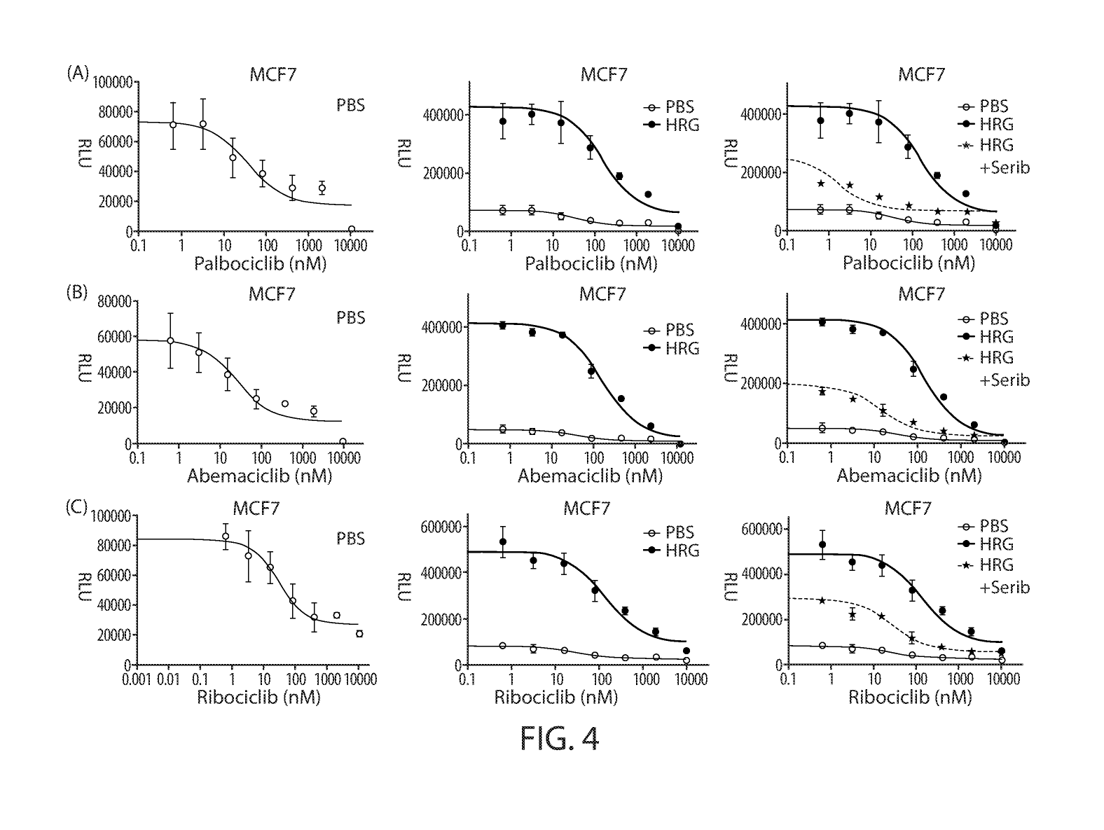

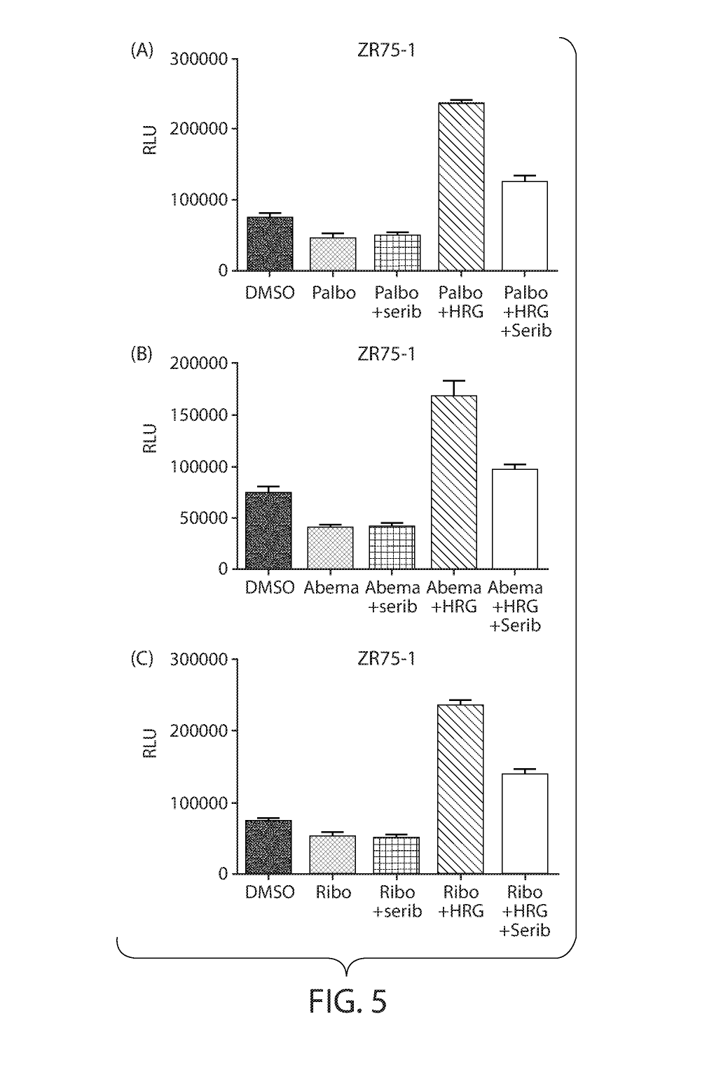

ER+, HER2− breast cancer cells were treated with CDK4/6 inhibitors in the absence or presence of HRG with or without the addition of seribantumab, followed by measurement of proliferation using the CTG assay (FIG. 4). MCF7 cells treated with single agent CDK4/6 inhibitors over a dose range (left most plots, A-C, FIG. 4) demonstrated that palbociclib, abemaciclib and ribociclib inhibited proliferation in a dose dependent manner and to a similar extent. MCF7 cells were also treated with each of the CDK4/6 inhibitors over the same dose range with a saturating dose of HRG (10 nM). HRG stimulation significantly repressed CDK4/6 inhibitor activity and increased proliferation (middle plots, A-C, FIG. 4). The addition of seribantumab restored CDK inhibitory activity (right hand plots, A-C, FIG. 4). Similar results were obtained in another ER+, HER2− cell line, ZR75-1, where HRG again inhibited the activity of CDK4/6 inhibitors and seribantumab restored sensitivity (FIGS. 5A-5C). In summary, these data indicate that HRG-ErbB3 signaling promotes insensitivity to the growth inhibitory effects of CDK4/6 inhibitors.

### Example 6: Heregulin Inhibits the Activity of the Combination of CDK4/6 Inhibitors and Endocrine Therapies in ER+, HER2− Metastatic Breast Cancer Cell Lines and Seribantumab Restores Sensitivity

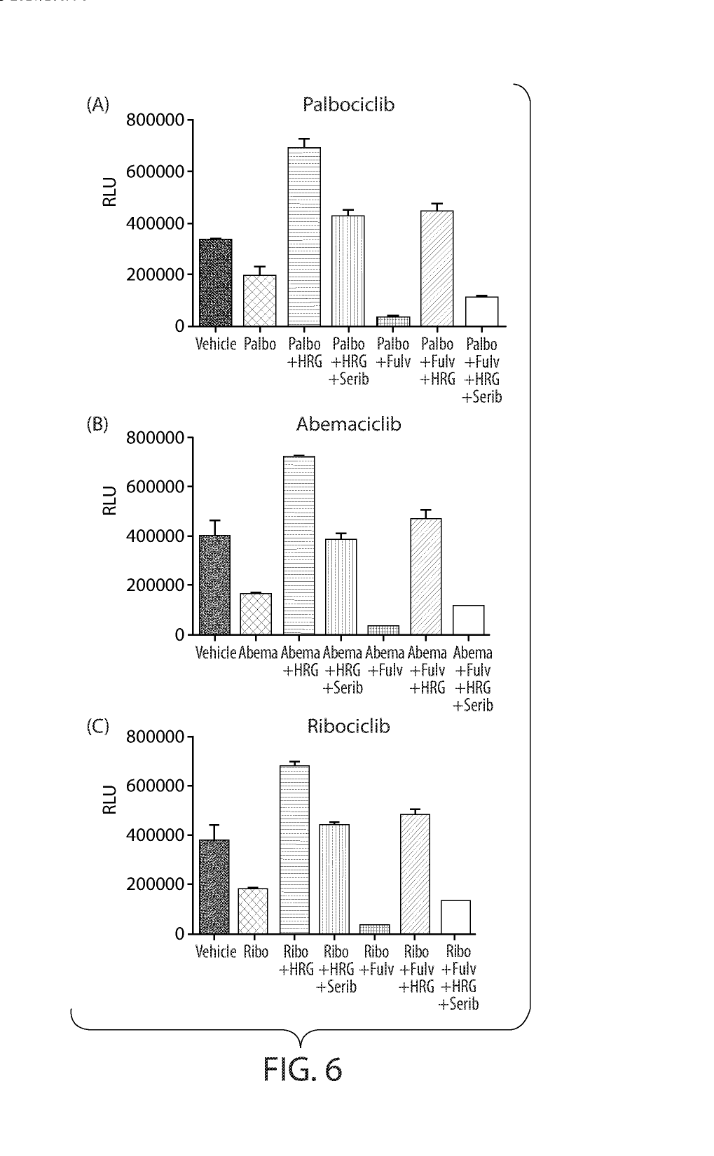

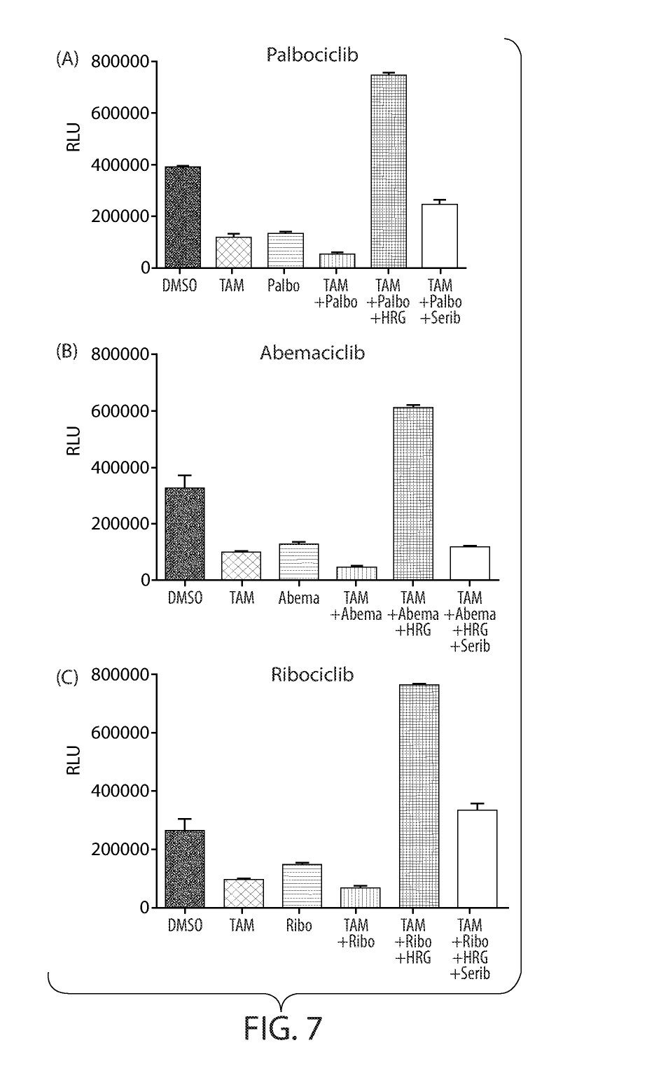

MCF7 cells were initially treated with various combinations of 1) palbociclib or abemaciclib or ribociclib, 2) HRG, 3) fulvestrant and 4) seribantumab, and proliferation was measured by CTG assay. When MCF7 cells were treated with the combination of a CDK4/6 inhibitor plus fulvestrant (50 nM), the degree of inhibition of proliferation was greater than the activity of the CDK4/6 inhibitor alone (FIGS. 6A-6C). Furthermore, for each CDK4/6 inhibitor-fulvestrant combination, the activity of this combination was blocked by the addition of HRG and seribantumab addition restored sensitivity to the CDK4/6 inhibitor-fulvestrant combination (FIGS. 6A-6C). The same experimental design was used to test matched combinations in which tamoxifen was substituted for fulvestrant, with similar results (FIGS. 7A-7C).

### Example 7: Treatment of ER+, HER2− Metastatic Breast Cancer with Palbociclib, a Hormonal Therapy, and Seribantumab in Patients not Previously Treated for Metastatic Breast Cancer

A patient with ER+, HER2− metastatic breast cancer is treated with one palbociclib 125 mg capsule taken orally once daily for 21 consecutive days, followed by 7 days off treatment to comprise a complete cycle of 28 days. The patient is concurrently treated with letrozole, 2.5 mg taken once daily continuously throughout the 28-day cycle, or with fulvestrant at a dose of 500 mg administered on days 1, 15, 29, and once monthly thereafter. The patient is also concurrently treated with seribantumab at a dose of 3 g every two weeks by IV infusion. Such treatment results in a beneficial result, e.g., stable disease, a partial response, or a complete response.

### Example 8: Treatment of ER+, HER2− Metastatic Breast Cancer with Palbociclib, a Hormonal Therapy, and Seribantumab in Patients Who have been Previously Treated with Palbociclib and a Hormonal Therapy, and Whose Cancer has Progressed on this Treatment

A patient with ER+, HER2− metastatic breast cancer who has been previously treated with palbociclib and either letrozole or fulvestrant and has become resistant to this treatment is treated with one palbociclib 125 mg capsule taken orally once daily for 21 consecutive days followed by 7 days off treatment to comprise a complete cycle of 28 days. The patient is concurrently treated with either letrozole (if the patient had been previously treated with fulvestrant) or fulvestrant (if the patient had been previously treated with letrozole). Letrozole is administered at a dose of 2.5 mg taken once daily continuously throughout the 28-day cycle, or fulvestrant is administered at a dose of 500 mg administered on days 1, 15, 29, and once monthly thereafter. The patient is also concurrently treated with seribantumab at a dose of 3 g every two weeks by IV infusion. Such treatment results in a beneficial result, e.g., stable disease, a partial response, or a complete response.

### Example 9: CDK2 Activation by Heregulin (HRG) Mitigates Fulvestrant or CDK4/6 Inhibitor Activity in HR+ HER2− Breast Cancer Cells and Seribantumab Restores Activity

The following experiments demonstrate that HER3 inhibitors can block non-canonical CDK2 complex by HRG in the presence of CDK4/6 inhibition by drugs, such as palbociclib, abemaciclib and ribociclib.

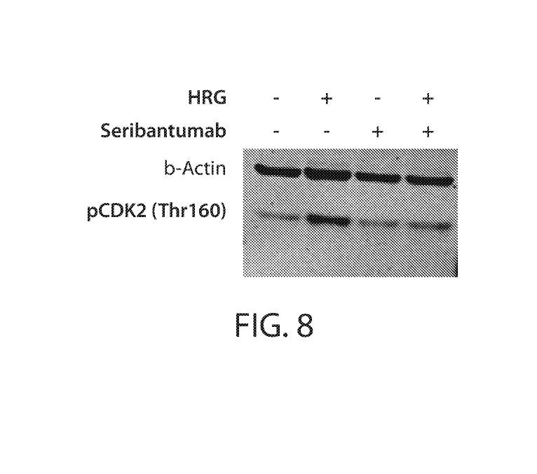

MCF7 cells were treated with 10 nM HRG, 100 nM fulvestrant, 100 nM palbociclib, 100 nM abemaciclib or 1 uM of seribantumab either alone or in combination for 20-24 hours as shown in FIGS. 8-10. Cellular lysates were prepared by lysis in MPER lysis buffer with the addition of protease and phosphatase inhibitors for 30 minutes on ice. Cellular debris was removed by centrifugation at 10,000 rpm. Proteins were analyzed by Western blotting according to standard protocols. Protein loading was estimated by blotting with a β-actin antibody (β-Actin (13E5) Rabbit mAb #4970 Cell Signaling Technology) and CDK2 activation was measured by detection of phosphorylation at threonine-160 of CDK2 with a pCDK2 antibody (Phospho-CDK2 (Thr160) Antibody #2561, Cell Signaling Technology).

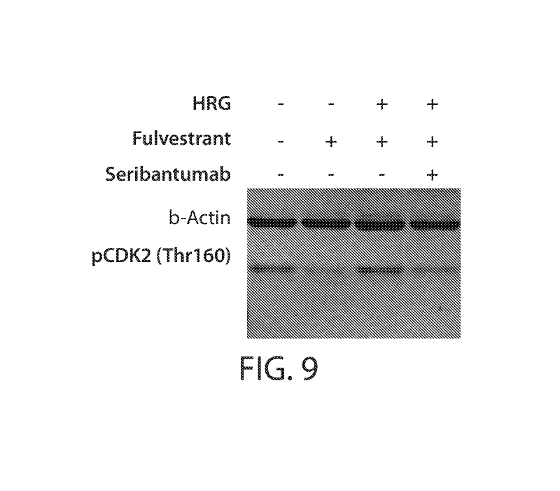

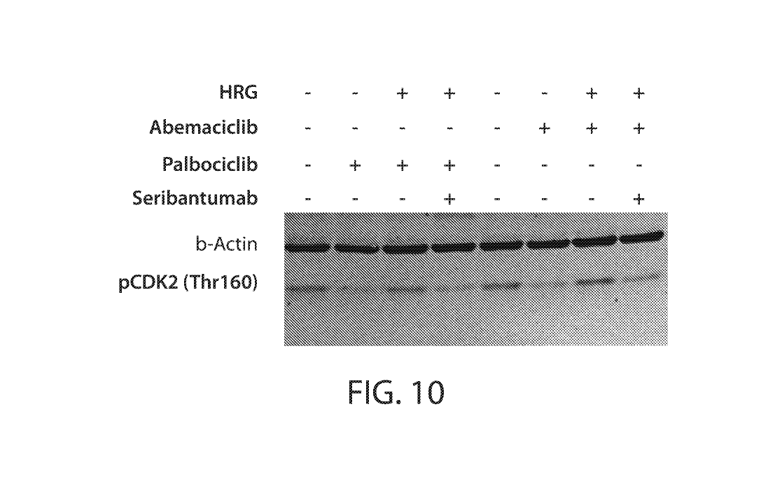

FIG. 8 demonstrates that HRG can activate CDK2 in HR+, HER2− breast cancer cells and that seribantumab can block the activating effect of HRG on CDK2 activation. Additionally, the anti-hormonal, fulvestrant and both of the CDK4/6 inhibitors, palbociclib or abemaciclib, inhibited CDK2 activation and HRG blocked this inhibitory activity (FIGS. 9 and 10). Moreover, seribantumab blocked HRG mediated activation of CDK2 in the presence of fulvestrant (FIG. 9) or CDK4/6 inhibitors palbociclib and abemaciclib (FIG. 10).

The implications of these findings are that one of the mechanisms by which HRG inhibits the activity of endocrine therapies (such as fulvestrant) and CDK4/6 inhibitors (such as palbociclib or abemaciclib) may be the non-canonical activation of CDK2 to promote cell cycle transition. These results suggest that seribantumab blocks this activation of CDK2 and therefore restores the activity of standard-of-care therapies, such as fulvestrant and CDK4/6 inhibitors.

### Example 10: HRG is a Highly Potent Ligand that Inhibits the Activities of Fulvestrant, Palbociclib and their Combination in the ER-Positive, HER2-Negative Breast Cancer Cells

Treatment of ER+ HER2− cell lines with multiple receptor tyrosine kinase ligands (RTKL) or estrogen (E2), illustrates that heregulin (HRG) is the most effective RTKL at inhibiting the activity of fulvestrant, palbociclib, or the combination of fulvestrant and palbociclib.

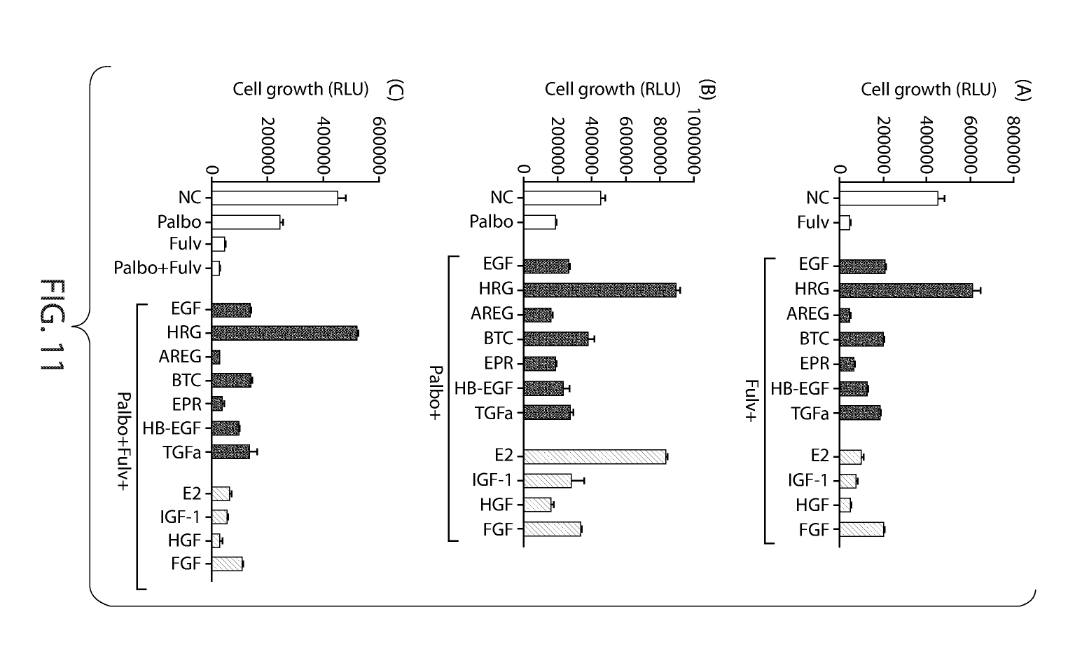

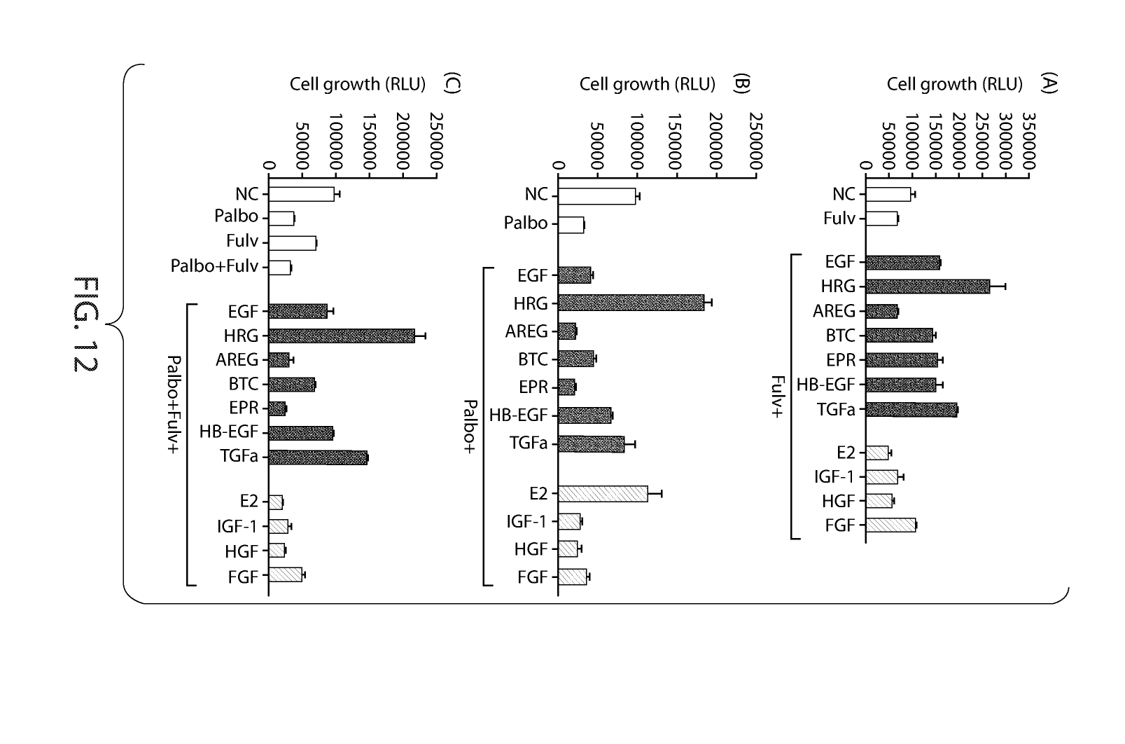

The purpose of this experiment was to determine if the observed effect of HRG on the activity of anti-estrogen therapies (e.g., fulvestrant) and CDK4-6 inhibitors (e.g., palbociclib) or their combinations is specific to HRG or if there is a broader effect that might be mediated by other growth promoting RTK ligands found in various cancers. To test this hypothesis, the ER+ HER2− cell lines MCF7 (FIG. 11) and T47D (FIG. 12) were treated with fulvestrant (50 nM), palbociclib (40 nM) as single agents or the combination of palbociclib plus fulvestrant (40 nM+50 nM) in the presence of the following ligands (concentration 1 nM) for 6 days at which time cellular growth was measured using the CTG assay.

EGF Family Ligands:
Epidermal growth factor (EGF)
Heregulin (HRG)
Amphiregulin (AREG)
Betacellulin (BTC)
Epiregulin (EPR)
Heparin-bound epidermal growth factor (HB-EGF)
Transforming growth factor alpha (TGFα)

Other Ligands:
Estradiol (E2)
Insulin-like growth factor 1 (IGF-1)
Hepatocyte growth factor (HGF)
FGF Basic (FGF2)

The data indicate that HRG is the most effective ligand out of all the ligands tested at inhibiting fulvestrant activity, palbociclib activity, and the combination of palbociclib and fulvestrant. These include both EGF family ligands and other ligands such as E2, IGF1 and FGF2.

### Example 11: HRG Promotes S-Phase Cell Cycle Progression of ER+ HER2− Cells. HRG Inhibits the Activity of Palbociclib in Combination with Fulvestrant on DNA Synthesis in ER+ Positive, HER2-Negative Breast Cancer Cells. Seribantumab Restores the Inhibitory Activity of this Combination

The objective of this experiment was to determine the effect of HRG on the activity of palbociclib and fulvestrant at the level of cell cycle progression. In addition, this experiment was designed to determine if seribantumab restores the cell cycle inhibitory activity of the individual components or the additive activity of a clinically approved drug combination by blocking the effect of HRG.

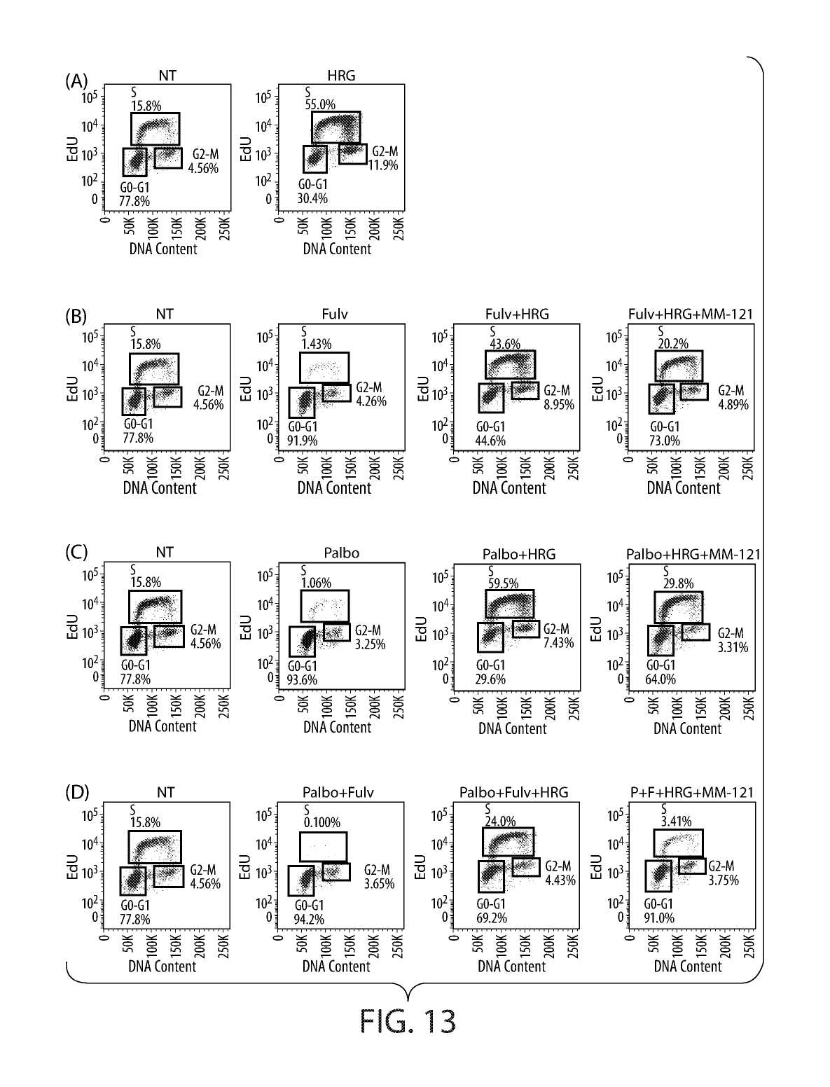

MCF7 cells were treated with combinations of palbociclib, fulvestrant, HRG and seribantumab for 24 hours, pulse-labelled with 10 μM EdU for 2 hours, fixed, double stained with Click-iT® EdU Alexa Fluor® 488 and FxCycle™ Violet stain and analyzed by flow cytometry. FIG. 13 is a representative FACS plot of the cell cycle distribution. The gate settings and percentage for cells in G0/G1, S and G2/M phases are indicated. DNA synthesis (S-phase) was determined by quantifying cells positive for both EdU incorporation and DNA content.

The data indicate that HRG can promote S-phase cell cycle progression of ER+ HER2− breast cancer cells (FIG. 13A) where the proportion of cells in the S-phase of resting cells was 15.8% and the proportion of S-phase cells in the HRG treated sample was 55%, indicating that HRG is capable of mediating DNA synthesis and cell cycle progression toward mitosis. Furthermore, treatment of the MCF7 cells with either fulvestrant (FIG. 13B) or palbociclib (FIG. 13C) as single agents in the absence of HRG demonstrates that both of these drugs are efficacious at inhibiting S-phase cell cycle progression, findings that are in agreement with the proposed mechanism of action for these drugs and the published literature. However, as shown in FIGS. 13A-13C, the cell cycle inhibitory activity of these drugs is significantly inhibited by the presence HRG and seribantumab restores this activity.

Finally, since both palbociclib and fulvestrant are commonly used in combination in ER+, HER2− breast cancer patients, the effect of HRG on the activity of the combination and whether seribantumab could restore this activity was tested. Consistent with our previous findings, HRG blocked the cell cycle inhibitory activity of the palbociclib-fulvestrant combination and seribantumab restored this activity (FIG. 13D). These findings are consistent with our other findings that HRG is capable of blocking the activity of clinically relevant and approved drug combinations for ER+ HER2− advanced breast cancer and that seribantumab restores this activity.

### Example 12: Seribantumab Enhances the Activity of Fulvestrant or the Combination of Fulvestrant and Palbociclib in Human Orthotopic Xenograft Models of ER+, HER2− Breast Cancer

The purpose of this experiment was to determine if the addition of seribantumab to either fulvestrant or palbociclib or their combination increases efficacy in an orthotopic xenograph model of ER+ HER2− breast cancer.

Female SHO mice (Charles River Laboratories) aged 8-10 weeks were implanted with 17-beta-estradiol pellets (0.72 mg/pellet) and 2 days later, injected with 100 μl of 5×10⁶ MCF7 cells suspended in 50% DPBS/50% matrigel into the abdominal mammary fat pad. Tumor growth was monitored twice a week and tumor volumes were calculated following external caliper measurement according to the formula (tumor size=π/6×[length×width²]). Once the average measured tumor volume had reached approximately 200 mm³, mice were randomized into groups and treatment was administered. Overall, the average starting tumor volume per group was equivalent across all groups. Fulvestrant was dosed at 500 μg per mouse once per week via subcutaneous delivery. Palbociclib was dosed at 25 mg/kg, orally every day for 5 days, Monday through Friday. Seribantumab was dosed a 600 μg per mouse twice per week via intraperitoneal injection.

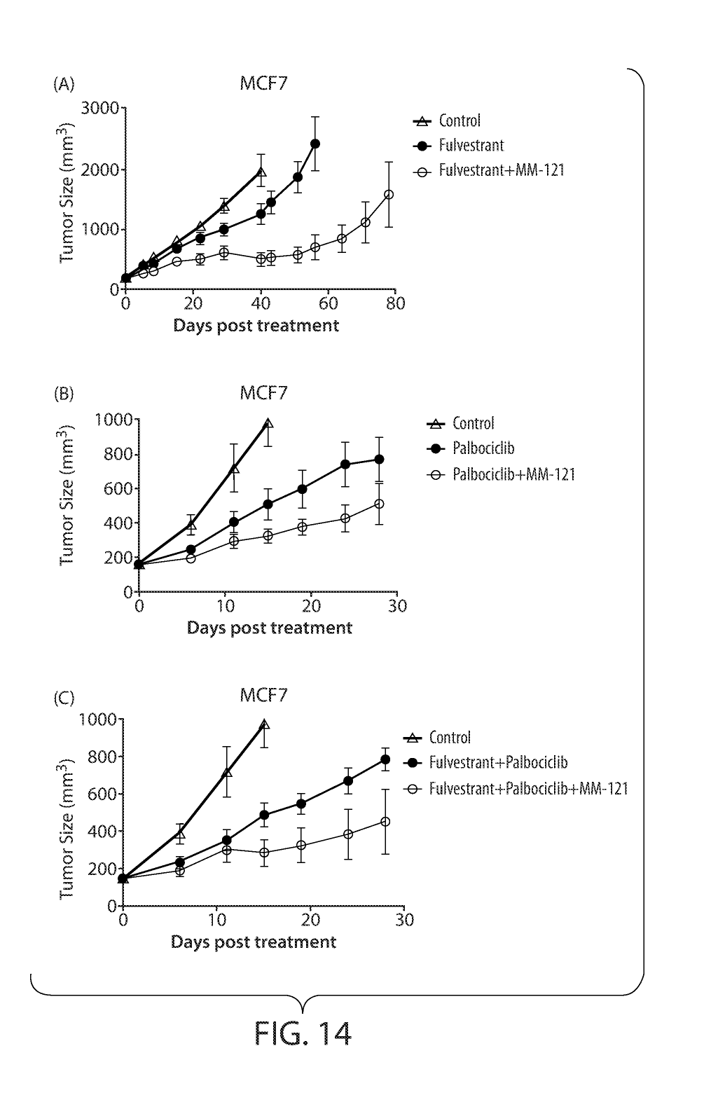

FIGS. 14A-14B show that the addition of seribantumab increased the antitumor efficacy of fulvestrant (FIG. 14A) and palbociclib (FIG. 14B) when either agent were used singularly. Furthermore, FIG. 14C shows that seribantumab increases the growth inhibition of the combination of palbociclib and fulvestrant.

This data is consistent with the broader in vitro findings that HRG can block the activity of anti-endocrine therapies such as tamoxifen or fulvestrant, CDK4-6 inhibitors (e.g., palbociclib, ribociclib or abemaciclib), and combinations thereof.

### Example 13: HRG Enhances the Phosphorylation of RB to Promote Cell Cycle Transition and Inhibit the Activity of Fulvestrant, CDK4/6 Inhibitors (e.g., Palbociclib or Abemaciclib) on RB Phosphorylation. Seribantumab can Restore Activity by Blockade of HRG in a Human ER+ HER2− Breast Cancer Cells

The purpose of this experiment was to examine the effect of HRG on the key cell cycle protein RB which is involved in mediating cell cycle progression via CDK4/6 activity. CDK4/6 inhibitors (e.g., palbociclib, ribociclib and abemaciclib) have a mechanism of action that is dependent on the cyclin D-CDK 4/6 complex and Rb protein. CDK4/6 inhibitors cause dephosphorylation the Rb protein, which represses transcription of the E2F gene and thus cell cycle inhibition.

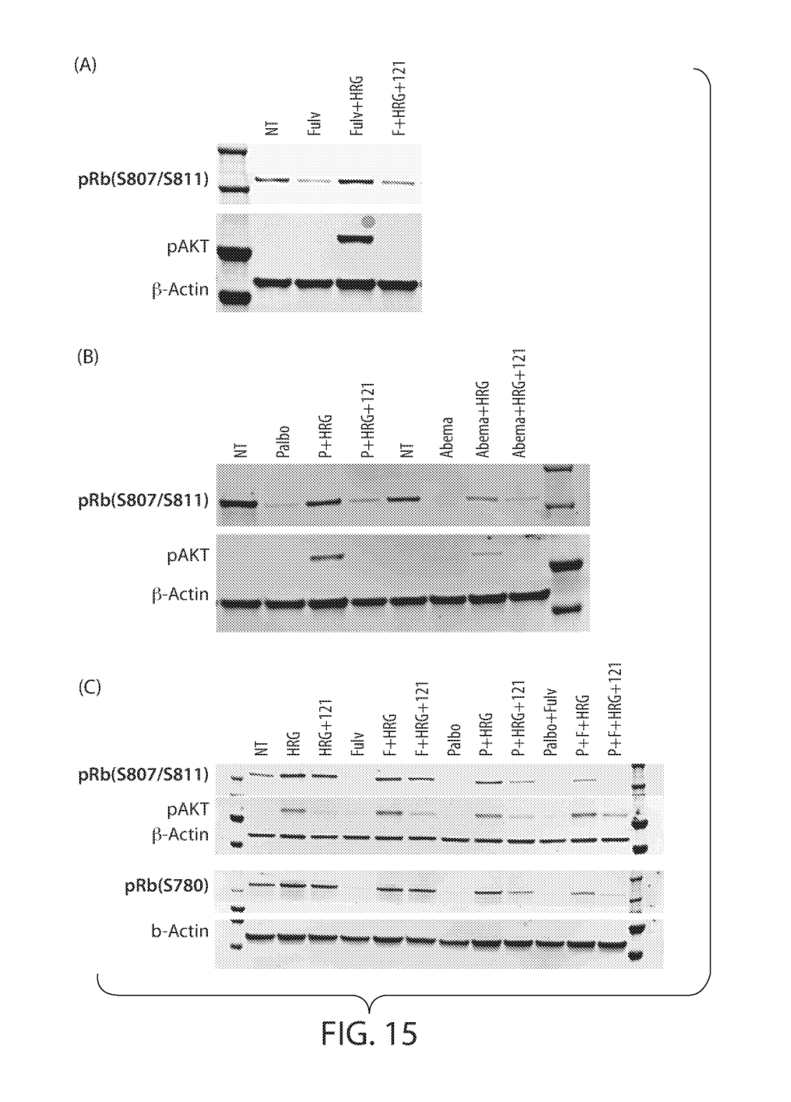

MCF7 cells were cultured as described above. Cells were treated with 10 nM HRG, 50 nM fulvestrant, 40 nM palbociclib, 40 nM abemaciclib or 1 uM of seribantumab either alone or in combination for 20-24 hours as shown in FIG. 15. Cellular lysates were prepared by lysis in MPER lysis buffer with the addition of protease and phosphatase inhibitors for 30 mins on ice. Cellular debris were removed by centrifugation at 10,000 rpm. Proteins were analyzed by Western blotting according to standard protocols. Protein loading was estimated by blotting with a β-actin antibody (β-Actin (13E5) Rabbit mAb #4970 Cell Signal) and RB activation was measured by detection of phosphorylation of RB (pRB) at pRb (S807/811): Cat #8516 pRb (S780): Cat #8180. Control antibodies as follows total AKT: Cat #9272 pAKT: Cat #4060.

FIG. 15 shows that HRG promoted the phosphorylation and activation of RB which counteracted the activity of fulvestrant and the CDK4/6 inhibitors, palbociclib and abemaciclib either alone or in combination. Furthermore, seribantumab restored the activity of fulvestrant and the CDK4/6 inhibitors, palbociclib and abemaciclib either alone or in combination.

### Example 14: Seribantumab and Letrozole Co-Treatment Delays the Onset of Resistance and Restores Sensitivity to Letrozole in MCF-7Ca Xenografts

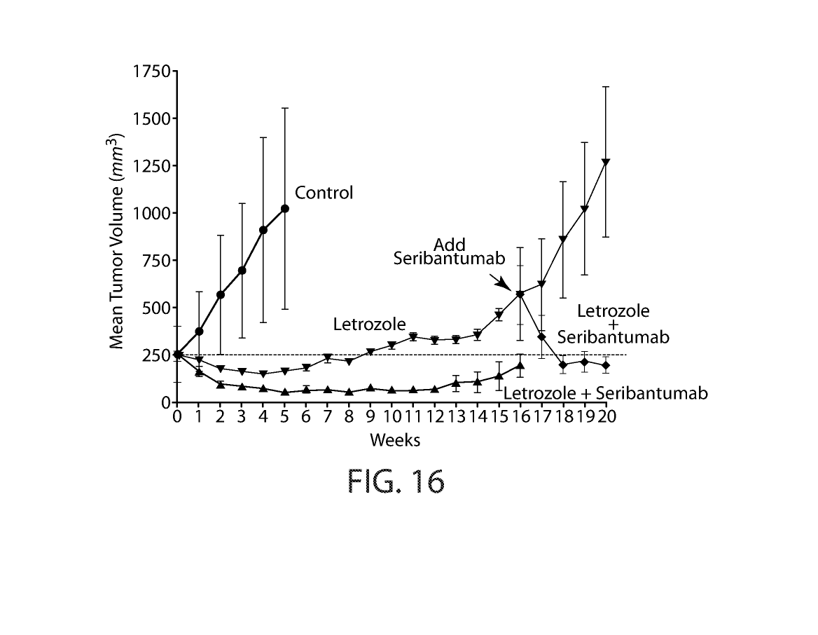

A model of post-menopausal ER+ breast cancer was used to determine the effect of blocking HRG-mediated ERB3 signaling and/or estrogen-mediated ER activation on tumor growth (FIG. 16). MCF-7Ca xenograft tumors were generated in female, ovariectomized nude mice, which were randomized to receive vehicle (“Control”; 0.3% hydroxypropylcellulose (HPC) in 0.9% NaCl, twice weekly (Q2W), intraperitoneal injection (IP); 15 mice/group), seribantumab (750 μg/mouse, Q2W, IP; 15 mice/group), letrozole (10 μg/mouse/day×5 days/week (QD×5), subcutaneous injection (SQ); 60 mice/group), or letrozole in combination with seribantumab, dosed as indicated for the monotherapies (15 mice/group). Changes in mean tumor volume (±SEM) were determined weekly by caliper measurement. Following the development of resistance to letrozole (week 14), mice in the letrozole-only group were re-randomized into 15 mice/group to receive: letrozole alone; seribantumab alone; or a combination of letrozole and seribantumab.

The MCF-7Ca-derived xenograft tumors initially responded to letrozole, but started to develop resistance after approximately 7-8 weeks of treatment (FIG. 16). When mice were co-treated with letrozole and seribantumab, however, tumor growth was inhibited and resistance to letrozole substantially delayed. This suggests that HRG/ERBB3 signaling was either active at the outset of the study or developed relatively quickly in response to letrozole treatment. Once resistance to letrozole was clearly established (week 14), mice in the letrozole-treated group were re-randomized to one of two cohorts: (i) continued letrozole monotherapy or (ii) seribantumab in combination with letrozole. Notably, the letrozole-resistant tumors displayed significantly decreased tumor growth when co-treated with letrozole and seribantumab compared to treatment with letrozole alone. This is consistent with the hypothesis that blocking both estrogen/ER- and HRG/ErbB33-driven signaling provides greater antitumor activity than blocking either pathway alone.

### Example 15: A Patient with ER/PR Positive, HER2 Negative, Locally Advanced or Metastatic Breast Cancer Whose Tumor Expresses HRG as Measured by RNA In-Situ Hybridization (RNA-ISH) is Given One of the Two Following Treatment Regimens

### Treatment A

Seribantumab: fixed dose of 3000 mg IV on days 1 and 15 of each 28-day cycle
Fulvestrant: 500 mg intramuscularly (IM) on days 1 and 15 of Cycle 1, and on Day 1 of each subsequent 28 day cycle

### Treatment B

Seribantumab: fixed dose of 3000 mg IV on days 1 and 15 of each 28-day cycle
Letrozole: 2.5 mg PO once per day

Such treatment regimens result in a beneficial result, e.g., stable disease, a partial response, or a complete response.

In some instances, the patient meets some or any of the following inclusion criteria:
a) Histologically or cytologically confirmed ER+ and/or PR+(with staining of >1% cells) breast cancer
b) Confirmed postmenopausal status due to either surgical/natural menopause or ovarian suppression (initiated at least 28 days prior to Day 1 of Cycle 1) with a gonadotropin-releasing hormone (GnRH) agonist such as goserelin
c) HER2 negative per ASCO/CAP guidelines
d) A positive in-situ hybridization (ISH) test for heregulin with a score of ≥1+, as determined by centralized testing of unstained tumor tissue
e) Must have at least one lesion amenable to either core needle biopsy or fine needle aspiration
f) Progressed following at least one but no more than three prior systemic therapies in the locally advanced or metastatic disease setting
   Received prior CDK inhibitor based therapy for locally advanced or metastatic disease
   Received no more than one prior line of chemotherapy for locally advanced or metastatic disease
g) Documented progression of locally advanced or metastatic disease as defined by RECIST v1.1. Exception: patients with bone-only metastatic disease are eligible if they have at least 2 lytic lesions visible on a CT or MRI and have documented disease progression on prior therapy based on the appearance of new lesions.
   Patients with bone-only lesions who have received radiation to those lesions must have documented progression following radiation therapy.
h) Able to understand and sign an informed consent (or have a legal representative who is able to do so)
i) ECOG Performance Score (PS) of 0 or 1
j) Adequate bone marrow reserves as evidenced by:
   ANC>1500/μl
   Platelet count>100,000/μl; and
   Hemoglobin>9 g/dL
k) Adequate hepatic function as evidenced by:
   Serum total bilirubin≤1.5×ULN except for patients with Morbus Gilbert
   Aspartate aminotransferase (AST), Alanine aminotransferase (ALT) and Alkaline
   Phosphatase≤2.5×ULN (≤5×ULN is acceptable if liver metastases are present, and ≤5×ULN of Alkaline Phosphatase is acceptable if bone metastases are present)
l) Adequate renal function as evidenced by a serum creatinine≤1.5×ULN
m) Recovered from clinically significant effects of any prior surgery, radiotherapy, or other antineoplastic therapy.
n) Patients may be treated with bone modifying agents such as bisphosphonates or receptor activator of nuclear factor kappa-B (RANK)-ligand agents (e.g. denosumab) per American Society of Clinical Oncology (ASCO) guidelines; whenever possible, patients requiring bone modifying agents should start treatment >7 days prior to study therapy and should continue the same agent throughout study unless clinically compelled to change
o) ≥18 years of age
p) Patients who have experienced a venous thromboembolic event within 60 days of signing the main consent form should have been treated with anti-coagulants for at least 7 days prior to beginning treatment on this study.

In some instances, the patient does not meet any of the following exclusion criteria:
a) Prior treatment with an anti-ErbB3 antibody
b) Prior treatment with fulvestrant in the locally advanced or metastatic setting c) Uncontrolled CNS disease or presence of leptomeningeal disease
d) History of another active malignancy that required systemic therapy in the last 2 years. Patients with prior history of in-situ cancer or basal or squamous cell skin cancer are eligible
e) Active infection, or an unexplained fever >38.5° C. during screening visits or on the first scheduled day of dosing, which in the investigator's opinion might compromise the patient's participation in the trial or affect the study outcome. At the discretion of the investigator, patients with tumor fever may be enrolled
f) Known hypersensitivity to any of the components of seribantumab, fulvestrant, or who have had hypersensitivity reactions to fully human monoclonal antibodies
g) Received other recent antitumor therapy including:
   Investigational therapy administered within the 28 days or 5 half-lives, whichever is shorter, prior to the first scheduled day of dosing in this study
   Radiation or other standard systemic therapy within 14 days prior to the first scheduled dose in this study, including, in addition (if necessary), the timeframe for resolution of any actual or anticipated toxicities from such radiation
h) NYHA Class III or IV congestive heart failure
i) Patients with a significant history of cardiac disease (i.e. uncontrolled blood pressure, unstable angina, myocardial infarction within 1 year or ventricular arrhythmias requiring medication) are also excluded
j) Uncontrolled infection requiring IV antibiotics, antivirals, or antifungals; or active human immunodeficiency virus (HIV) infection, active hepatitis B infection or active hepatitis C infection
k) Any other medical condition deemed by the Investigator to be likely to interfere with a patient's ability to sign informed consent, interfere with a patient's ability to cooperate and participate in the study, or interfere with the interpretation of the results

### SEQUENCE SUMMARY

| SEQ ID | | | |
|---|---|---|---|
| NO: | DESIGNATION | SEQUENCE | |
| 1 | Heavy Chain | His Tyr Val Met Ala | |
| | CDR1 (CDRH1) | | |
| | of seribantumab | | |
| | Protein | | |
| 2 | Heavy Chain | Ser Ile Ser Ser Ser Gly Gly Trp Thr Leu | |
| | CDR2 (CDRH2) | Tyr Ala Asp Ser Val Lys Gly | |
| | of seribantumab | | |
| | Protein | | |
| 3 | Heavy Chain | Gly Leu Lys Met Ala Thr Ile Phe Asp Tyr | |
| | CDR3 (CDRH3) | | |
| | of seribantumab | | |
| | Protein | | |
| 4 | Light Chain | Thr Gly Thr Ser Ser Asp Val Gly Ser Tyr | |
| | CDR1 (CDRL1) | Asn Val Val Ser | |
| | of seribantumab | | |
| | Protein | | |
| 5 | Light Chain | Glu Val Ser Gln Arg Pro Ser | |
| | CDR2 (CDRL2) | | |
| | of seribantumab | | |
| | Protein | | |
| 6 | Light Chain | Cys Ser Tyr Ala Gly Ser Ser Ile Phe Val | |
| | CDR3 (CDRL3) | Ile | |
| | of seribantumab | | |
| | Protein | | |
| 7 | Heavy Chain of | 1 | EVQLLESGGG LVQPGGSLRL SCAASGFTFS HYVMAWVRQA PGKGLEWVSS |
| | Antibody | 51 | ISSSGGWTLY ADSVKGRFTI SRDNSKNTLY LQMNSLRAED TAVYYCTRGL |
| | seribantumab | 101 | KMATIFDYWG QGTLVTVSSA STKGPSVFPL APCSRSTSES TAALGCLVKD |
| | Protein | 151 | YFPEPVTVSW NSGALTSGVH TFPAVLQSSG LYSLSSVVTV PSSNFGTQTY |
| | | 201 | TCNVDHKPSN TKVDKTVERK CCVECPPCPA PPVAGPSVFL FPPKPKDTLM |
| | | 251 | ISRTPEVTCV VVDVSHEDPE VQFNWYVDGV EVHNAKTKPR EEQFNSTFRV |
| | | 301 | VSVLTVVHQD WLNGKEYKCK VSNKGLPAPI EKTISKTKGQ PREPQVYTLP |
| | | 351 | PSREEMTKNQ VSLTCLVKGF YPSDIAVEWE SNGQPENNYK TTPPMLDSDG |
| | | 401 | SFFLYSKLTV DKSRWQQGNV FSCSVMHEAL HNHYTQKSLS LSPGK |
| 8 | Light Chain of | 1 | QSALTQPASV SGSPGQSITI SCTGTSSDVG SYNVVSWYQQ HPGKAPKLII |
| | seribantumab | 51 | YEVSQRPSGV SNRFSGSKSG NTASLTISGL QTEDEADYYC CSYAGSSIFV |
| | Protein | 101 | lFGGGTKVTV LGQPKAAPSV TLFPPSSEEL QANKATLVCL VSDFYPGAVT |
| | | 151 | VAWKADGSPV KVGVETTKPS KQSNNKYAAS SYLSLTPEQW KSHRSYSCRV |
| | | 201 | THEGSTVEKT VAPAECS |
| 9 | Heavy Chain | gaggtgcagc tgctggagag cggcggaggg | |
| | Variable Region | ctggtccagc caggcggcag cctgaggctg | |
| | (VH) of | tcctgcgccg ccagcggctt caccttcagc | |
| | seribantumab | cactacgtga tggcctgggt gcggcaggcc | |
| | DNA | ccaggcaagg gcctggaatg ggtgtccagc | |
| | | atcagcagca gcggcggctg gaccctgtac | |
| | | gccgacagcg tgaagggcag gttcaccatc | |
| | | agcagggaca acagcaagaa caccctgtac | |
| | | ctgcagatga acagcctgag ggccgaggac | |
| | | accgccgtgt actactgcac caggggcctg | |
| | | aagatggcca ccatcttcga ctactggggc | |
| | | cagggcaccc tggtgaccgt gagcagc | |
| 10 | Heavy Chain | Glu Val Gln Leu Leu Glu Ser Gly Gly Gly | |
| | Variable Region | Leu Val Gln Pro Gly Gly Ser Leu Arg Leu | |
| | (VH) of | Ser Cys Ala Ala Ser Gly Phe Thr Phe Ser | |
| | seribantumab | His Tyr Val Met Ala Trp Val Arg Gln Ala | |
| | Protein | Pro Gly Lys Gly Leu Glu Trp Val Ser Ser | |
| | | Ile Ser Ser Ser Gly Gly Trp Thr Leu Tyr | |
| | | Ala Asp Ser Val Lys Gly Arg Phe Thr Ile | |
| | | Ser Arg Asp Asn Ser Lys Asn Thr Leu Tyr | |
| | | Leu Gln Met Asn Ser Leu Arg Ala Glu Asp | |
| | | Thr Ala Val Tyr Tyr Cys Thr Arg Gly Leu | |
| | | Lys Met Ala Thr Ile Phe Asp Tyr Trp Gly | |
| | | Gln Gly Thr Leu Val Thr Val Ser Ser | |
| 11 | Light Chain | cagtccgccc tgacccagcc cgccagcgtg | |
| | Variable Region | agcggcagcc caggccagag catcaccatc | |
| | (VL) of | agctgcaccg gcaccagcag cgacgtgggc | |
| | seribantumab | agctacaacg tggtgtcctg gtatcagcag | |
| | DNA | caccccggca aggcccccaa gctgatcatc | |
| | | tacgaggtgt cccagaggcc cagcggcgtg | |
| | | agcaacaggt tcagcggcag caagagcggc | |
| | | aacaccgcca gcctgaccat cagcggcctg | |
| | | cagaccgagg acgaggccga ctactactgc | |
| | | tgcagctacg ccggcagcag catcttcgtg | |
| | | atcttcggcg gagggaccaa ggtgaccgtc cta | |
| 12 | Light Chain | Gln Ser Ala Leu Thr Gln Pro Ala Ser Val | |
| | Variable Region | Ser Gly Ser Pro Gly Gln Ser Ile Thr Ile | |
| | (VL) of | Ser Cys Thr Gly Thr Ser Ser Asp Val Gly | |
| | seribantumab | Ser Tyr Asn Val Val Ser Trp Tyr Gln Gln | |
| | Protein | His Pro Gly Lys Ala Pro Lys Leu Ile Ile | |
| | | Tyr Glu Val Ser Gln Arg Pro Ser Gly Val | |
| | | Ser Asn Arg Phe Ser Gly Ser Lys Ser Gly | |
| | | Asn Thr Ala Ser Leu Thr Ile Ser Gly Leu | |
| | | Gln Thr Glu Asp Glu Ala Asp Tyr Tyr Cys | |
| | | Cys Ser Tyr Ala Gly Ser Ser Ile Phe Val | |
| | | Ile Phe Gly Gly Gly Thr Lys Val Thr Val | |
| | | Leu | |
| 13 | HRG cDNA | 1 | gaggccaggg gagggtgcga aggaggcgcc tgcctccaac ctgcgggcgg gaggtgggtg |
| | (GenBank | 61 | gctgcggggc aattgaaaaa gagccggcga ggagttcccc gaaacttgtt ggaactccgg |
| | accession number | 121 | gctcgcgcgg aggccaggag ctgagcggcg gcggctgccg gacgatggga gcgtgagcag |
| | NM-013956) | 181 | gacggtgata acctctcccc gatcgggttg cgagggcgcc gggcagaggc caggacgcga |
| | | 241 | gccgccagcg gtgggaccca tcgacgactt cccggggcga caggagcagc cccgagagcc |
| | | 301 | agggcgagcg cccgttccag gtggccggac cgcccgccgc gtccgcgccg cgctccctgc |
| | | 361 | aggcaacggg agacgccccc gcgcagcgcg agcgcctcag cgcggccgct cgctctcccc |
| | | 421 | ctcgagggac aaacttttcc caaacccgat ccgagccctt ggaccaaact cgcctgcgcc |
| | | 481 | gagagccgtc cgcgtagagc gctccgtctc cggcgagatg tccgagcgca aagaaggcag |
| | | 541 | aggcaaaggg aagggcaaga agaaggagcg aggctccggc aagaagccgg agtccgcggc |
| | | 601 | gggcagccag agcccagcct tgcctccccg attgaaagag atgaaaagcc aggaatcggc |
| | | 661 | tgcaggttcc aaactagtcc ttcggtgtga aaccagttct gaatactcct ctctcagatt |
| | | 721 | caagtggttc aagaatggga atgaattgaa tcgaaaaaac aaaccacaaa atatcaagat |
| | | 781 | acaaaaaaag ccagggaagt cagaacttcg cattaacaaa gcatcactgg ctgattctgg |
| | | 841 | agagtatatg tgcaaagtga tcagcaaatt aggaaatgac agtgcctctg ccaatatcac |
| | | 901 | catcgtggaa tcaaacgaga tcatcactgg tatgccagcc tcaactgaag gagcatatgt |
| | | 961 | gtcttcagag tctcccatta gaatatcagt atccacagaa ggagcaaata cttcttcatc |
| | | 1021 | tacatctaca tccaccactg ggacaagcca tcttgtaaaa tgtgcggaga aggagaaaac |
| | | 1081 | tttctgtgtg aatggagggg agtgcttcat ggtgaaagac ctttcaaacc cctcgagata |
| | | 1141 | cttgtgcaag tgcccaaatg agtttactgg tgatcgctgc caaaactacg taatggccag |
| | | 1201 | cttctacaag catcttggga ttgaatttat ggaggcggag gagctgtacc agaagagagt |
| | | 1261 | gctgaccata accggcatct gcatcgccct ccttgtggtc ggcatcatgt gtgtggtggc |
| | | 1321 | ctactgcaaa accaagaaac agcggaaaaa gctgcatgac cgtcttcggc agagccttcg |
| | | 1381 | gtctgaacga aacaatatga tgaacattgc caatgggcct caccatccta acccaccccc |
| | | 1441 | cgagaatgtc cagctggtga atcaatacgt atctaaaaac gtcatctcca gtgagcatat |
| | | 1501 | tgttgagaga gaagcagaga catccttttc caccagtcac tatacttcca cagcccatca |
| | | 1561 | ctccactact gtcacccaga ctcctagcca cagctggagc aacggacaca ctgaaagcat |
| | | 1621 | cctttccgaa agccactctg taatcgtgat gtcatccgta gaaaacagta ggcacagcag |
| | | 1681 | cccaactggg ggcccaagag gacgtcttaa tggcacagga ggccctcgtg aatgtaacag |
| | | 1741 | cttcctcagg catgccagag aaacccctga ttcctaccga gactctcctc atagtgaaag |
| | | 1801 | gtatgtgtca gccatgacca ccccggctcg tatgtcacct gtagatttcc acacgccaag |
| | | 1861 | ctcccccaaa tcgccccctt cggaaatgtc tccacccgtg tccagcatga cggtgtccat |
| | | 1921 | gccttccatg gcggtcagcc ccttcatgga agaagagaga cctctacttc tcgtgacacc |
| | | 1981 | accaaggctg cgggagaaga agtttgacca tcaccctcag cagttcagct ccttccacca |
| | | 2041 | caaccccgcg catgacagta acagcctccc tgctagcccc ttgaggatag tggaggatga |
| | | 2101 | ggagtatgaa acgacccaag agtacgagcc agcccaagag cctgttaaga aactcgccaa |
| | | 2161 | tagccggcgg gccaaaagaa ccaagcccaa tggccacatt gctaacagat tggaagtgga |
| | | 2221 | cagcaacaca agctcccaga gcagtaactc agagagtgaa acagaagatg aaagagtagg |
| | | 2281 | tgaagatacg cctttcctgg gcatacagaa ccccctggca gccagtcttg aggcaacacc |
| | | 2341 | tgccttccgc ctggctgaca gcaggactaa cccagcaggc cgcttctcga cacaggaaga |
| | | 2401 | aatccaggcc aggctgtcta gtgtaattgc taaccaagac cctattgctg tataaaacct |
| | | 2461 | aaataaacac atagattcac ctgtaaaact ttattttata taataaagta ttccacctta |
| | | 2521 | aattaaacaa tttattttat tttagcagtt ctgcaaatag aaaacaggaa aaaaactttt |
| | | 2581 | ataaattaaa tatatgtatg taaaaatgtg ttatgtgcca tatgtagcaa ttttttacag |
| | | 2641 | tatttcaaaa cgagaaagat atcaatggtg cctttatgtt atgttatgtc gagagcaagt |
| | | 2701 | tttgtacagt tacagtgatt gcttttccac agtatttctg caaaacctct catagattca |
| | | 2761 | gtttttgctg gcttcttgtg cattgcatta tgatgttgac tggatgtatg atttgcaaga |
| | | 2821 | cttgcaactg tccctctgtt tgcttgtagt agcacccgat cagtatgtct tgtaatggca |
| | | 2881 | catccatcca gatatgcctc tcttgtgtat gaagttttct ttgctttcag aatatgaaat |
| | | 2941 | gagttgtgtc tactctgcca gccaaaggtt tgcctcattg ggctctgaga taatagtaga |
| | | 3001 | tccaacagca tgctactatt aaatacagca agaaactgca ttaagtaatg ttaaatatta |
| | | 3061 | ggaagaaagt aatactgtga tttaaaaaaa act |

| | | |
|---|---|---|
| 14 | ErbB3 | SEVGNSQAVCPGTLNGLSVTGDAENQYQTLYKLYERCEVV |
| | protein | MGNLEIVLTGHNADLSFLQWIREVTGYVLVAMNEFSTLPLPN |
| | | LRVVRGTQVYDGKFAIFVMLNYNTNSSHALRQLRLTQLTEIL |
| | | SGGVYIEKNDKLCHMDTIDWRDIVRDRDAEIVVKDNGRSCP |
| | | PCHEVCKGRCWGPGSEDCQTLTKTICAPQCNGHCFGPNPNQ |
| | | CCHDECAGGCSGPQDTDCFACRHFNDSGACVPRCPQPLVYN |
| | | KLTFQLEPNPHTKYQYGGVCVASCPHNFVVDQTSCVRACPP |
| | | DKMEVDKNGLKMCEPCGGLCPKACEGTGSGSRFQTVDSSNI |
| | | DGFVNCTKILGNLDFLITGLNGDPWHKIPALDPEKLNVFRTV |
| | | REITGYLNIQSWPPHMHNFSVFSNLTTIGGRSLYNRGFSLLIM |
| | | KNLNVTSLGFRSLKEISAGRIYISANRQLCYHHSLNWTKVLR |
| | | GPTEERLDIKHNRPRRDCVAEGKVCDPLCSSGGCWGPGPGQ |
| | | CLSCRNYSRGGVCVTHCNFLNGEPREFAHEAECFSCHPECQP |
| | | MEGTATCNGSGSDTCAQCAHFRDGPHCVSSCPHGVLGAKGP |
| | | IYKYPDVQNECRPCHENCTQGCKGPELQDCLGQTLVLIGKTH |
| | | LTMALTVIAGLVVIFMMLGGTFLYWRGRRIQNKRAMRRYLE |
| | | RGESIEPLDPSEKANKVLARIFKETELRKLKVLGSGVFGTVHK |
| | | GVWIPEGESIKIPVCIKVIEDKSGRQSFQAVTDHMLAIGSLDH |
| | | AHIVRLLGLCPGSSLQLVTQYLPLGSLLDHVRQHRGALGPQL |
| | | LLNWGVQIAKGMYYLEEHGMVHRNLAARNVLLKSPSQVQV |
| | | ADFGVADLLPPDDKQLLYSEAKTPIKWMALESIHFGKYTHQS |
| | | DVWSYGVTVWELMTFGAEPYAGLRLAEVPDLLEKGERLAQ |
| | | PQICTIDVYMVMVKCWMIDENIRPTFKELANEFTRMARDPPR |
| | | YLVIKRESGPGIAPGPEPHGLTNKKLEEVELEPELDLDLDLEA |
| | | EEDNLATTTLGSALSLPVGTLNRPRGSQSLLSPSSGYMPMNQ |
| | | GNLGESCQESAVSGSSERCPRPVSLHPMPRGCLASESSEGHV |
| | | TGSEAELQEKVSMCRSRSRSRSPRPRGDSAYHSQRHSLLTPV |
| | | TPLSPPGLEEEDVNGYVMPDTHLKGTPSSREGTLSSVGLSSVL |
| | | GTEEEDEDEEYEYMNRRRRHSPPHPPRPSSLEELGYEYMDVG |
| | | SDLSASLGSTQSCPLHPVPIMPTAGTTPDEDYEYMNRQRDGG |
| | | GPGGDYAAMGACPASEQGYEEMRAFQGPGHQAPHVHYARL |
| | | KTLRSLEATDSAFDNPDYWHSRLFPKANAQRT |

1. A method of treating a patient with metastatic ER+, HER2− HRG+ breast cancer, the method comprising concurrently administering to the patient:
I) one palbociclib 125 mg capsule taken orally once daily for 21 consecutive days followed by 7 days off treatment to comprise a complete cycle of 28 days;
II) either a) or b) wherein a) is letrozole, 2.5 mg given once daily continuously throughout the 28-day cycle, and b) is fulvestrant administered at a dose of 500 mg on days 1, 15, 29, and once monthly thereafter; and
I) seribantumab at a dose of 3 g every two weeks by IV infusion.

2. The method of claim 1, wherein the patient is identified as HRG+ if a heregulin RNA in situ hybridization (RNA-ISH) score of 1+ of higher has been measured in a biological sample of a tumor from the patient prior to treatment.

3. The method of claim 1, wherein treatment results in the patient exhibiting stable disease, a partial response, or a complete response.

4. A method of treating a patient with metastatic ER+, HER2− HRG+ breast cancer who has been previously treated with palbociclib and a hormonal therapy, and whose cancer has progressed on this treatment, the method comprising concurrently administering to the patient:
I) one palbociclib 125 mg capsule taken orally once daily for 21 consecutive days followed by 7 days off treatment to comprise a complete cycle of 28 days;
II) either a) or b) wherein a) is letrozole, 2.5 mg given once daily continuously throughout the 28-day cycle, and b) is fulvestrant administered at a dose of 500 mg on days 1, 15, 29, and once monthly thereafter and wherein if the patient previously was treated with fulvestrant, then the patent is administered a) and if the patient was previously treated with letrozole, then the patent is administered b); and
I) seribantumab at a dose of 3 g every two weeks by IV infusion.

5. The method of claim 4, wherein the patient is identified as HRG+ if a heregulin RNA in situ hybridization (RNA-ISH) score of 1+ of higher has been measured in a biological sample of a tumor from the patient prior to treatment.

6. The method of claim 4, wherein treatment results in the patient exhibiting stable disease, a partial response, or a complete response.

7. A method of treating a patient with ER+, HER2− HRG+ breast cancer, the method comprising concurrently administering to the patient: (i) a non-steroidal aromatase inhibitor or selective estrogen receptor degrader; (ii) an anti-ErbB3 antibody; and, optionally (iii) a CDK4/6 inhibitor.

8. A method of treating a patient with ER/PR+, HER2− breast cancer expressing HRG as measured by RNA in-situ hybridization (RNA-ISH), the method comprising a 28-day cycle, wherein:
I) seribantumab is administered at a dose of 3000 mg intravenously (IV) on days 1 and 15 of the cycle, and
II) fulvestrant is administered at a dose of 500 mg intramuscularly (IM) on days 1 and 15 of the cycle.

9. The method of claim 8, wherein the method comprises at least one subsequent treatment cycle.

10. The method of claim 9, wherein fulvestrant is administered only on day 1 of each subsequent treatment cycle.

11. A method of treating a patient with ER/PR+, HER2− breast cancer expressing HRG as measured by RNA in-situ hybridization (RNA-ISH), the method comprising at least one 28-day cycle, wherein:
I) seribantumab is administered at a dose of 3000 mg IV on days 1 and 15 of the cycle, and
II) letrozole is administered at a dose of 2.5 mg orally once per day during the cycle.

12. The method of claim 8, wherein the breast cancer is locally advanced or metastatic breast cancer.


SEQUENCE LISTING (13 sequences)

SEQ ID NO: 1 - protein, 5 aa, Artificial Sequence
  source: /note=“Description of Artificial Sequence      Synthetic peptide”
HYVMA

SEQ ID NO: 2 - protein, 17 aa, Artificial Sequence
  source: /note=“Description of Artificial Sequence      Synthetic peptide”
SISSSGGWTLYADSVKG

SEQ ID NO: 3 - protein, 10 aa, Artificial Sequence
  source: /note=“Description of Artificial Sequence      Synthetic peptide”
GLKMATIFDY

SEQ ID NO: 4 - protein, 14 aa, Artificial Sequence
  source: /note=“Description of Artificial Sequence      Synthetic peptide”
TGTSSDVGSYNVVS

SEQ ID NO: 5 - protein, 7 aa, Artificial Sequence
  source: /note=“Description of Artificial Sequence      Synthetic peptide”
EVSQRPS

SEQ ID NO: 6 - protein, 11 aa, Artificial Sequence
  source: /note=“Description of Artificial Sequence      Synthetic peptide”
CSYAGSSIFVI

SEQ ID NO: 7 - protein, 445 aa, Artificial Sequence
  source: /note=“Description of Artificial Sequence      Synthetic polypeptide”
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 217 aa, Artificial Sequence
  source: /note=“Description of Artificial Sequence      Synthetic polypeptide”
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 9 - DNA, 357 bp, Artificial Sequence
  source: /note=“Description of Artificial Sequence      Synthetic polynucleotide”
gaggtgcagctgctggagagcggcggagggctggtccagccaggcggcagcctgaggctgtcctgcgccgccagcggcttcaccttcagccactacgtgatggcctgggtgcggcaggccccaggcaagggcctggaatgggtgtccagcatcagcagcagcggcggctggaccctgtacgccgacagcgtgaagggcaggttcaccatcagcagggacaacagcaagaacaccctgtacctgcagatgaacagcctgagggccgaggacaccgccgtgtactactgcaccaggggcctgaagatggccaccatcttcgactactggggccagggcaccctggtgaccgtgagcagc

SEQ ID NO: 10 - protein, 119 aa, Artificial Sequence [sequence omitted]

SEQ ID NO: 11 - DNA, 333 bp, Artificial Sequence
  source: /note=“Description of Artificial Sequence      Synthetic polynucleotide”
cagtccgccctgacccagcccgccagcgtgagcggcagcccaggccagagcatcaccatcagctgcaccggcaccagcagcgacgtgggcagctacaacgtggtgtcctggtatcagcagcaccccggcaaggcccccaagctgatcatctacgaggtgtcccagaggcccagcggcgtgagcaacaggttcagcggcagcaagagcggcaacaccgccagcctgaccatcagcggcctgcagaccgaggacgaggccgactactactgctgcagctacgccggcagcagcatcttcgtgatcttcggcggagggaccaaggtgaccgtccta

SEQ ID NO: 12 - protein, 111 aa, Artificial Sequence [sequence omitted]

SEQ ID NO: 13 - DNA, 3093 bp, Homo sapiens
gaggccaggggagggtgcgaaggaggcgcctgcctccaacctgcgggcgggaggtgggtggctgcggggcaattgaaaaagagccggcgaggagttccccgaaacttgttggaactccgggctcgcgcggaggccaggagctgagcggcggcggctgccggacgatgggagcgtgagcaggacggtgataacctctccccgatcgggttgcgagggcgccgggcagaggccaggacgcgagccgccagcggtgggacccatcgacgacttcccggggcgacaggagcagccccgagagccagggcgagcgcccgttccaggtggccggaccgcccgccgcgtccgcgccgcgctccctgcaggcaacgggagacgcccccgcgcagcgcgagcgcctcagcgcggccgctcgctctccccctcgagggacaaacttttcccaaacccgatccgagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccgattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacaagcatcttgggattgaatttatggaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaaaacttttataaattaaatatatgtatgtaaaaatgtgttatgtgccatatgtagcaattttttacagtatttcaaaacgagaaagatatcaatggtgcctttatgttatgttatgtcgagagcaagttttgtacagttacagtgattgcttttccacagtatttctgcaaaacctctcatagattcagtttttgctggcttcttgtgcattgcattatgatgttgactggatgtatgatttgcaagacttgcaactgtccctctgtttgcttgtagtagcacccgatcagtatgtcttgtaatggcacatccatccagatatgcctctcttgtgtatgaagttttctttgctttcagaatatgaaatgagttgtgtctactctgccagccaaaggtttgcctcattgggctctgagataatagtagatccaacagcatgctactattaaatacagcaagaaactgcattaagtaatgttaaatattaggaagaaagtaatactgtgatttaaaaaaaact